(19) United States
(12) Patent Application Publication
(10) Pub. No.: US 2025/0213526 A1
(43) Pub. Date: Jul. 3, 2025

(54) TRIPLE UPTAKE INHIBITOR FOR THE TREATMENT OF ATYPICAL DEPRESSION

(71) Applicant: Noema Pharma AG, Basel (CH)

(72) Inventor: George Garibaldi, Basel (CH)

(21) Appl. No.: 18/841,749

(22) PCT Filed: Feb. 28, 2023
(86) PCT No.: PCT/EP2023/055052

§ 371 Date: Aug. 27, 2024

Related U.S. Application Data
(60) Provisional application No. 63/484,905, filed on Feb. 14, 2023.
(60) Provisional application No. 63/314,753, filed on Feb. 28, 2022.

(51) Int. Cl. A61K 31/40; A61P 3/04; A61P 25/24

(52) U.S. Cl. CPC A61K 31/40; A61P 3/04; A61P 25/24

(57) ABSTRACT

Provided herein are methods of treating atypical depression in a subject in need thereof by administering to the subject compositions comprising a triple reuptake inhibitor, having the structure of Compound (1).

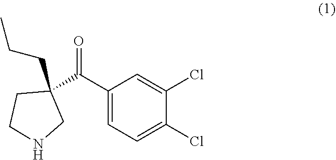 [SMILES: CCC[C@]1(C(=O)c2ccc(Cl)c(Cl)c2)CCNC1]

### CROSS-REFERENCE TO RELATED APPLICATION

The application claims the benefit of, and priority to, U.S. Ser. No. 63/314,753 filed on Feb. 28, 2022, and U.S. Ser. No. 63/484,905 filed on Feb. 14, 2023, the contents of each of which are incorporated herein by reference.

### BACKGROUND

Atypical depression is a subtype of depression which is characterized by mood reactivity and atypical symptoms including weight gain or hyperphagia, hypersomnia, leaden paralysis, and interpersonal rejection sensitivity resulting in significant social or occupational impairment. Atypical depression is the most common form of depression seen in outpatient clinics in psychiatry. Current literature supports atypical depression as a subtype of depression with high prevalence, early onset in life, and tendency to persist longer. Patients of atypical depression with chronic course, pattern of long-standing rejection sensitivity, and the always present fatigue can easily end up with primary diagnosis of personality disorder or simply neurosis. This can have profound adverse effects if it results in denial of a trial of antidepressants, which have been found to be very effective.

Approximately 30% of the patients with unipolar depression meet atypical depression criteria. Yet atypical depression is poorly studied, and patients meeting the criteria for this subtype are often excluded from major depression trials. Currently, despite numerous drug trials, there are no comprehensive treatment guidelines for atypical depression. Patients with atypical depression have shown a preferential response to phenelzine sulfate compared with imipramine hydrochloride in placebo-controlled trials, prompting the recommendation of monoamine oxidase inhibitors (MAOIs) as the standard of care. Unfortunately, MAOIs have dietary restrictions, contraindications, and well-known side effects.

Accordingly, there is an unmet medical need for new methods that address treating individuals suffering from atypical depression.

### SUMMARY

Provided herein, in part, are methods of treating a subject manifesting at least one or more behaviors selected from the group consisting of: (a) (i) mood reactivity, (ii) increased sleep, (iii) hypersomnia, (iv) leaden paralysis, (v) long-standing pattern of interpersonal rejection sensitivity resulting in significant social or occupational impairment, (b) (i) binge eating not associated with the regular use of inappropriate compensatory behaviors, (ii) binge eating not occurring exclusively during the course of anorexia nervosa or bulimia nervosa, (c) (i) recurrent inappropriate compensatory behaviors, such as self-induced vomiting; misuse of laxatives, diuretics, or other medications, in order to prevent weight gain, (ii) binge eating and inappropriate compensatory behaviors, (iii) self-evaluation is unjustifiability influenced by body shape and weight, (iv) disturbance not occurring exclusively during episodes of anorexia nervosa, (v) purging type of recurrent compensatory behavior, and (vi) non-purging type of recurrent compensatory behavior, (d) (i) recurrent episodes of binge eating, (ii) eating in a discrete period of time an amount of food that is definitely larger than most people would eat in a similar period of time under similar circumstances, (iii) a sense of lack of control over eating during the episode, (iv) a feeling that one cannot stop eating or control what or how much one is eating, (v) eating much more rapidly than normal, (vi) eating until feeling uncomfortably full, (vii) eating large amounts of food when not feeling physically hungry, (viii) eating alone because of feeling embarrassed by how much one is eating, (ix) feeling disgusted with oneself, depressed or very guilty after overeating, (x) marked distress regarding binge eating, (e) (i) increased appetite, (ii) hyperphagia, (iii) significant lack of impulse control followed by guilt feelings, (f) (i) restriction of energy intake relative to requirements leading to a significantly low body weight, (ii) intense fear of gaining weight or becoming fat, even though underweight, and (iii) disturbance in the way in which one's body weight or shape is experienced; said method comprising administering a composition comprising Compound 1:

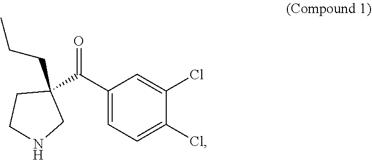 [SMILES: CCC[C@]1(C(=O)c2ccc(Cl)c(Cl)c2)CCNC1]

or a pharmaceutically acceptable salt thereof, sufficient to improve at least one of the behaviors.

Also provided herein is a method of treating a subject manifesting an impulsive behavior, comprising administering a pharmaceutical composition comprising Compound 1:

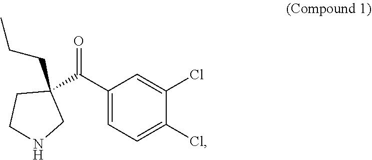 [SMILES: CCC[C@]1(C(=O)c2ccc(Cl)c(Cl)c2)CCNC1]

or a pharmaceutically acceptable salt thereof.

In another aspect, also provided herein is a method of treating a subject who has been diagnosed with atypical depression, comprising administering a pharmaceutical composition comprising Compound 1:

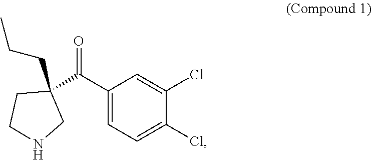 [SMILES: CCC[C@]1(C(=O)c2ccc(Cl)c(Cl)c2)CCNC1]

or a pharmaceutically acceptable salt thereof, wherein the subject manifests one or more behavior selected from the group consisting of: hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight.

In another aspect, also provided herein is a method of treating a subject manifesting at least one or more of a behavior selected from the group consisting of: hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight; said method comprising administering a unit dose comprising Compound 1:

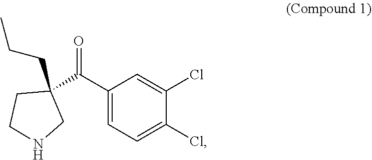 [SMILES: CCC[C@]1(C(=O)c2ccc(Cl)c(Cl)c2)CCNC1]

or a pharmaceutically acceptable salt thereof, sufficient to reduce at least of said behaviors.

Also provided herein is a method of treating Prader-Willi syndrome in a subject in need thereof, comprising administering a composition comprising Compound 1:

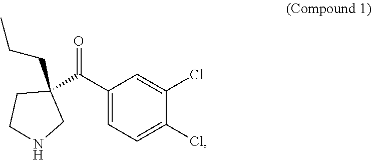 [SMILES: CCC[C@]1(C(=O)c2ccc(Cl)c(Cl)c2)CCNC1]

or a pharmaceutically acceptable salt thereof.

### BRIEF DESCRIPTION OF THE DRAWINGS

FIG. 1 depicts an exemplary X-ray powder diffraction pattern of a hydrochloride quarterhydrate of (3,4-dichloro-phenyl)-((S)-3-propyl-pyrrolidin-3-yl)-methanone.

FIG. 2 depicts an exemplary X-ray powder diffraction pattern of a hydrochloride of (3,4-dichloro-phenyl)-((S)-3-propyl-pyrrolidin-3-yl)-methanone.

FIG. 3 depicts a mean (±SEM) of chow intake (in grams) by adult male Sprague Dawley rats (n=31) during a 30-minute session, where **p<0.001: significant between groups effect of treatment on chow consumption between Compound 1 and vehicle (VEH).

FIG. 4 depicts a mean (±SEM) of total lever presses by adult male Sprague Dawley rats (n=31) during a 30-minute session, where **p<0.001: significant difference between Compound 1 treatments and vehicle (VEH) on lever presses.

FIG. 5 depicts a mean (±SEM) of total lever presses by adult male Sprague Dawley rats (n=31) during a 30-minute operant session, where **p<0.001: significant difference between Compound 1 treatments (n=16) and vehicle (VEH) (n=15) on lever presses.

FIG. 6. depicts a mean (±SEM) of latency to the first 6 continuous epochs of nonrapid eye movement sleep (NR).

FIG. 7. depicts a mean (±SEM) of latency to the first 3 continuous epochs of rapid eye movement sleep (REM).

FIG. 8 and FIG. 9 depict effect of Compound 1 on chocolate intake. FIGS. 8 and 9 illustrate that Compound 1 is effective in controlling binge eating in animal model.

FIG. 10A and FIG. 10B depict the five-choice serial-reaction time task (5CSRTT) test in rats to study impulsive behaviors in rats. FIGS. 10A and 10B illustrate that Compound 1 reduces impulsive behaviors in rats and that Compound 1 dose-dependently reduced premature and perseverative responses with no adverse effects on accuracy or speed of responding.

FIG. 11 depicts an SR™ model of Compound 1 plasma concentration versus SERT occupancy in Raphe Nuclei as a result of a PET study in HVs. FIG. 11 illustrates that there is 70% to 90% SERT occupancy at 30 mg to 60 mg of Compound 1.

FIG. 12 depicts a 2TCM model of Compound 1 plasma concentration versus DAT occupancy in the striatum. FIG. 12 illustrates that there is 25% to 40% DAT occupancy at 30 mg to 60 mg of Compound 1.

FIG. 13 depicts mean (±SEM) intake of chocolate over the 12 1-hour training sessions during the chocolate intake experiment described in Example 7.

FIG. 14 and FIG. 15 depict effects of Compound 1 on lever pressing and chow consumption of high (n=15) and low (n=16) responder groups in PROG/Chow feeding choice task. FIG. 14 depicts a mean (±SEM) number of total lever presses during a 30-minute session, where **p<0.001: significant difference between Compound 1 dose treatments and VEH on lever presses. FIG. 15 depicts a mean (±SEM) of chow intake (in grams) during a 30-minute session, where **p<0.001, *p<0.01: significant difference between groups effect of treatment on chow consumption between dose Compound 1 and VEH.

### DETAILED DESCRIPTION

As generally described herein, the present disclosure provides methods of treating atypical depression in a subject in need thereof. The methods comprise administering to a subject in need thereof a composition comprising a therapeutically effective amount of Compound 1 or a pharmaceutically acceptable salt thereof. In addition, the present disclosure provides methods of treating atypical depression with a crystalline form of either Compound 1 or a pharmaceutically acceptable salt thereof.

### Methods of Use and Treatment

In one aspect, provided herein are methods of treating atypical depression in a subject in need thereof. In another aspect, provided herein are methods of treating a medical condition associated with atypical depression in a subject in need thereof.

In one aspect, provided herein are methods of treating a subject manifesting at least one or more behaviors selected from the group consisting of: (a) (i) mood reactivity, (ii) increased sleep, (iii) hypersomnia, (iv) leaden paralysis, (v) long-standing pattern of interpersonal rejection sensitivity resulting in significant social or occupational impairment, (b) (i) binge eating not associated with the regular use of inappropriate compensatory behaviors, (ii) binge eating not occurring exclusively during the course of anorexia nervosa or bulimia nervosa, (c) (i) recurrent inappropriate compensatory behaviors, such as self-induced vomiting; misuse of laxatives, diuretics, or other medications, in order to prevent weight gain, (ii) binge eating and inappropriate compensatory behaviors, (iii) self-evaluation is unjustifiability influenced by body shape and weight, (iv) disturbance not occurring exclusively during episodes of anorexia nervosa, (v) purging type of recurrent compensatory behavior, and (vi) non-purging type of recurrent compensatory behavior, (d) (i) recurrent episodes of binge eating, (ii) eating in a discrete period of time an amount of food that is definitely larger than most people would eat in a similar period of time under similar circumstances, (iii) a sense of lack of control over eating during the episode, (iv) a feeling that one cannot stop eating or control what or how much one is eating, (v) eating much more rapidly than normal, (vi) eating until feeling uncomfortably full, (vii) eating large amounts of food when not feeling physically hungry, (viii) eating alone because of feeling embarrassed by how much one is eating, (ix) feeling disgusted with oneself, depressed or very guilty after overeating, (x) marked distress regarding binge eating, (e) (i) increased appetite, (ii) hyperphagia, (iii) significant lack of impulse control followed by guilt feelings, (f) (i) restriction of energy intake relative to requirements leading to a significantly low body weight, (ii) intense fear of gaining weight or becoming fat, even though underweight, and (iii) disturbance in the way in which one's body weight or shape is experienced; said method said method comprising administering a composition comprising Compound 1;

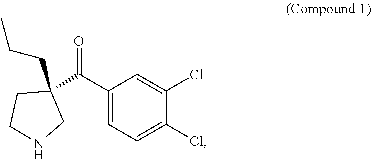 [SMILES: CCC[C@]1(C(=O)c2ccc(Cl)c(Cl)c2)CCNC1]

or a pharmaceutically acceptable salt thereof, sufficient to improve at least one of the behaviors. Each behavior described herein is described in the Diagnostic and Statistical Manual of Mental Disorder, for example in the Diagnostic and Statistical Manual of Mental Disorder, 5^{th} Edition.

In some embodiments, the composition is a pharmaceutical composition.

In some embodiments, the pharmaceutical composition is a tablet.

In some embodiments, the pharmaceutical composition is a sustained release form.

In some embodiments, the pharmaceutical composition is a unit dose.

In some embodiments, the composition comprises from about 1 mg to about 70 mg of Compound 1, or a pharmaceutically acceptable salt thereof. In some embodiments, the composition comprises from about 1 mg to about 60 mg of Compound 1. In some embodiments, the composition comprises from about 1 mg to about 45 mg of Compound 1. In some embodiments, the composition comprises from about 1 mg to about 30 mg of Compound 1. In some embodiments, the composition comprises from about 1 mg to about 15 mg of Compound 1. In some embodiments, the composition comprises from about 1 mg to about 5 mg of Compound 1. In some embodiments, the composition comprises about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, or about 70 mg of Compound 1. In some embodiments, the composition comprises from about 10 mg of Compound 1. In some embodiments, the composition comprises from about 15 mg of Compound 1. In some embodiments, the composition comprises from about 20 mg of Compound 1. In some embodiments, the composition comprises from about 30 mg of Compound 1. In some embodiments, the composition comprises from about 35 mg of Compound 1.

In some embodiments, the composition comprises from about 1 mg/kg to about 70 mg/kg of Compound 1, or a pharmaceutically acceptable salt thereof. In some embodiments, the composition comprises from about 1 mg/kg to about 60 mg/kg of Compound 1. In some embodiments, the composition comprises from about 1 mg/kg to about 45 mg/kg of Compound 1. In some embodiments, the composition comprises from about 1 mg/kg to about 30 mg/kg of Compound 1. In some embodiments, the composition comprises from about 1 mg/kg to about 15 mg/kg of Compound 1. In some embodiments, the composition comprises from about 1 mg/kg to about 5 mg/kg of Compound 1. In some embodiments, the composition comprises about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg of Compound 1.

In some embodiments, the composition is administered orally.

In some embodiments, the composition is administered once daily.

In some embodiments, said subject manifests mood reactivity; and at least two behaviors selected from the group consisting of increased appetite, hyperphagia, increased sleep, hypersomnia, leaden paralysis, and long-standing pattern of interpersonal rejection sensitivity resulting in significant social or occupational impairment. In embodiments, said subject does not meet the criteria for melancholic depression or catatonia.

In some embodiments, said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In an embodiment, said subject manifests mood reactivity and said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In an embodiment, said subject manifests hyperphagia and said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In an embodiment, said subject manifests hypersomnia and said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In an embodiment, said subject manifests leaden paralysis and said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In an embodiment, said subject manifests long-standing pattern of interpersonal rejection sensitivity resulting in significant social or occupational impairment and said subject further manifests one or more behaviors selected from the group consisting of recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

In some embodiments, said subject manifests recurrent episodes of binge eating, eating in a discrete period of time an amount of food that is definitely larger than most people would eat in a similar period of time under similar circumstances, a sense of lack of control over eating during the episode, eating much more rapidly than normal, a feeling that one cannot stop eating or control what or how much one is eating, marked distress regarding binge eating, binge eating not associated with the regular use of inappropriate compensatory behaviors, and binge eating not occurring exclusively during the course of anorexia nervosa or bulimia nervosa; and three or more behaviors selected from the group consisting of eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating. In some embodiments, said subject manifests distress regarding binge eating. In some embodiments, the binge eating occurs, on average, at least once a week for 3 months. In some embodiments, the binge eating is not associated with the regular use of inappropriate compensatory behaviors and does not occur exclusively during the course of anorexia nervosa or bulimia nervosa. Inappropriate compensatory behaviors include, but not limited to, purging, fasting, and excessive exercise.

In some embodiments, said subject manifests recurrent episodes of binge eating, eating in a discrete period of time an amount of food that is definitely larger than most people would eat in a similar period of time under similar circumstances, a feeling that one cannot stop eating or control what or how much one is eating, recurrent inappropriate compensatory behaviors in order to prevent weight gain, binge eating and inappropriate compensatory behaviors, self-evaluation is unjustifiability influenced by body shape and weight, and disturbance not occurring exclusively during episodes of anorexia nervosa. In some embodiments, the discrete period of time is a 2-hour period, and the binge eating and inappropriate compensatory behaviors occur at least twice a week for 3 months.

In some embodiments, said subject is a subject who has been diagnosed with atypical depression.

In some embodiments, said subject is a subject who has been diagnosed with one or more behaviors selected from the group consisting of mood reactivity, increased appetite, hyperphagia, increased sleep, hypersomnia, leaden paralysis, and long-standing pattern of interpersonal rejection sensitivity resulting in significant social or occupational impairment.

In some embodiments, said subject is a subject who has been diagnosed with binge eating disorder. In some embodiments, said subject is a subject who has been diagnosed with one or more behaviors selected from the group consisting of recurrent episodes of binge eating, eating in a discrete period of time an amount of food that is definitely larger than most people would eat in a similar period of time under similar circumstances, a sense of lack of control over eating during the episode, eating much more rapidly than normal, a feeling that one cannot stop eating or control what or how much one is eating, marked distress regarding binge eating, binge eating not associated with the regular use of inappropriate compensatory behaviors, binge eating not occurring exclusively during the course of anorexia nervosa or bulimia nervosa, eating much more rapidly than normal, eating until feeling uncomfortably full, eating large amounts of food when not feeling physically hungry, eating alone because of feeling embarrassed by how much one is eating, and feeling disgusted with oneself, depressed or very guilty after overeating.

In some embodiments, said subject is a subject who has been diagnosed with bulimia nervosa. In some embodiments, said subject is a subject who has been diagnosed with one or more behaviors selected from the group consisting of rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, and non-purging type of recurrent compensatory behavior.

In some embodiments, said subject is a subject who has been diagnosed with anorexia nervosa. In some embodiments, said subject is a subject who has been diagnosed with one or more behaviors selected from the group consisting of restriction of energy intake relative to requirements leading to a significantly low body weight in the context of age, sex, developmental trajectory, and physical health, intense fear of gaining weight or becoming fat, even though underweight, and disturbance in the way in which one's body weight or shape is experienced, undue influence of body weight or shape on self-evaluation, or denial of the seriousness of the current low body weight.

In some embodiments, said subject is a subject who has been diagnosed with depression.

In some embodiments, said subject is a subject who has been treated for depression.

In some embodiments, the method comprises treating a subject who has been diagnosed with depression and also manifests one or more atypical depression symptoms. In embodiments, said atypical depression symptom is selected from the group consisting of mood reactivity, increased appetite, hyperphagia, increased sleep, hypersomnia, leaden paralysis, and long-standing pattern of interpersonal rejection sensitivity resulting in significant social or occupational impairment.

In some embodiments, the manifestation of the behavior occurs at least once daily.

In some embodiments, the manifestation of the behavior persists for at least a week. In some embodiments, the manifestation of the behavior persists for at least two weeks. In some embodiments, the manifestation of the behavior persists for at least three weeks.

In some embodiments, the manifestation of the behavior persists for at least a month. In some embodiments, the manifestation of the behavior persists for at least three months.

In some embodiments, manifestation of behavior occurs periodically.

Also provided herein is a method of treating a subject manifesting an impulsive behavior, comprising administering a pharmaceutical composition comprising Compound 1:

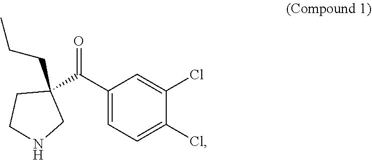 [SMILES: CCC[C@]1(C(=O)c2ccc(Cl)c(Cl)c2)CCNC1]

or a pharmaceutically acceptable salt thereof. In certain embodiments, the impulsive behavior is characterized by a significant lack of impulse control followed by guilt feelings.

In another aspect, also provided herein is a method of treating a subject who has been diagnosed with atypical depression, comprising administering a pharmaceutical composition comprising Compound 1:

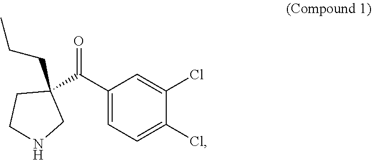 [SMILES: CCC[C@]1(C(=O)c2ccc(Cl)c(Cl)c2)CCNC1]

or a pharmaceutically acceptable salt thereof, wherein the subject manifests one or more behavior selected from the group consisting of: hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight.

In another aspect, also provided herein is a method of treating a subject manifesting at least one or more of a behavior selected from the group consisting of: hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight; said method comprising administering a unit dose comprising Compound 1:

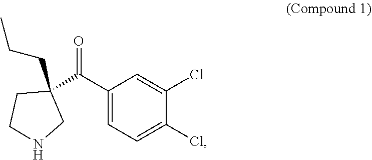 [SMILES: CCC[C@]1(C(=O)c2ccc(Cl)c(Cl)c2)CCNC1]

or a pharmaceutically acceptable salt thereof, sufficient to reduce at least of said behaviors.

Also provided herein is a method of treating Prader-Willi syndrome in a subject in need thereof, comprising administering a composition comprising Compound 1:

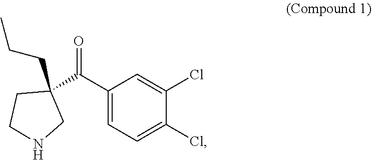 [SMILES: CCC[C@]1(C(=O)c2ccc(Cl)c(Cl)c2)CCNC1]

or a pharmaceutically acceptable salt thereof.

In an aspect, Compound 1 is a therapeutic agent, and the method comprises administering to a subject in need thereof a composition comprising a therapeutically effective amount of the therapeutic agent. In embodiments, the therapeutic efficacy of the treatment is determined by assessing improvement based on a depressive symptom scale (for example, Inventory of Depressive Symptoms—Self-Reported (IDS-SR) or a modified version thereof). In embodiments, the therapeutic efficacy of the treatment is determined by assessing improvement based on the Mini International Neuropsychiatric Interview (MINI-S). In embodiments, the therapeutic efficacy of the treatment is determined by assessing improvement based on the Clinical Global Impression—Severity (CGI-S). In embodiments, the therapeutic efficacy of the treatment is determined by assessing improvement based on the Epworth Sleepiness Scale (ESS) and total sleep time (TST; measured by actigraphy). In embodiments, the therapeutic efficacy of the treatment is determined by assessing improvement based on the Clinical Global Impression—Improvement (CGI-I). In embodiments, the therapeutic efficacy of the treatment is determined by assessing improvement based on the Patient Global Impression of Change (PGI-C). In embodiments, the therapeutic efficacy of the treatment is determined by assessing improvement based on a fatigue scale (for example, the Brief Fatigue Inventory (BFI), the Fatigue Severity Scale (FSS), the Fatigue Symptom Inventory (FSI), the Fatigue—Short Form 10a (FACIT-Fatigue-10)). In various embodiments, the subject is determined to be much improved. In various embodiments, the subject is determined to be very much improved. In various embodiments, the subject shows a change from baseline.

### Triple Reuptake Inhibitor

A triple reuptake inhibitor is a serotonin-norepinephrine-dopamine reuptake inhibitor that acts as a combined reuptake inhibitor of the monoamine neurotransmitters serotonin, norepinephrine, and dopamine. A triple reuptake inhibitor concomitantly inhibits the serotonin transporter (SERT), norepinephrine transporter (NET), and dopamine transporter (DAT), respectively. Inhibition of the reuptake of these neurotransmitters increases their extracellular concentrations and, therefore, results in an increase in serotonergic, adrenergic, and dopaminergic neurotransmission across the brain. One assay to measure affinity of compounds of the present disclosure may be the percent inhibition from a radioligand binding assay using cells expressing human transporters and receptors. Relevant radioligand binding assays to determine affinity are well-known in the art (See Pacholczyk, T. et. al., 1991, Nature 350, 350-354; Pristupa, Z. et al., 1994, Mol. Pharmacol. 45, 125-135; Tatsumi, M. et. al., 1999, Eur. J. Pharmacol. 368, 277-283)

### Atypical Depression

Atypical depression is a subtype of major depression disorders (MDD) characterized by mood reactivity (moods that are strongly reactive to environmental circumstances and feeling extremely sensitive—this is a must have feature), hypersomnia, carbohydrate craving/increased appetite, leaden paralysis (profound fatigue), and chronic rejection sensitivity. Atypical depression results in more disability than melancholic depression, because individuals often have more interpersonal difficulties. Behaviours distinct to patients with atypical depression include a mood that temporarily brightens after a positive event or happy news and heavy feelings in arms and legs. Patients with atypical depression are usually excluded from MDD development programs. Individuals with atypical depression are 26% of MDD patients, and prevalence is approximately 1-4% in the general population. Criteria heavily gender specific (women are more prone to develop AD symptoms). Atypical depression is also associated with conduct disorder, social phobia, interpersonal dependency, low self-esteem, and parental substance abuse. Atypical depression is also associated with higher rates of early childhood trauma, whereas melancholic depression is not.

Distinct Characteristics and Neurobiology of AD include early onset and low suicidality, more likeliness to run a chronic course with multiple co-morbidities, association with low HPA axis activity but is not with catecholaminergic abnormalities, phenotype similar to hypercortisolism (Cushing) and hypothyroidism, and appearance of sleep architecture close to normal despite hypersomnia and increased sleep duration (>10 hours per day).

Atypical depression as a separate diagnosis was introduced primarily because medication trials clearly showed such patients responded better to monoamine oxidase inhibitors (MAOIs) compared to tricyclic antidepressants (TCAs). Currently, both primary care providers and psychiatrists are involved in MDD patient journey, however, Psychiatrists are the only ones capable of diagnosing atypical depression.

Atypical feature as a specifier can be applied to major depressive episodes (MDD), dysthymia, nonbipolar disorder, bipolar disorder when a major depressive episode is the most recent mood episode. According to Diagnostic and Statistical Manual of Mental Disorders V (DSM-5), in part of symptoms of MDD, five or more symptoms during the same 2 weeks, of which one is at least: (1) depressed mood most of the day; and (2) diminished interest or pleasure in daily activities.

The following criteria are required to make the diagnosis of atypical depression according to DSM-5. In addition to meeting the criteria for major depressive disorder, the following specifier criteria may be required to make the diagnosis of atypical depression.
A. Mood reactivity (i.e.—mood brightens in response to actual or potential positive events)
B. 2 or more of the following:
   (1) Significant weight gain or increase in appetite
   (2) Hypersomnia
   (3) Leaden paralysis (i.e.—heavy, leaden feelings in arms or legs)
   (4) A long-standing pattern of interpersonal rejection sensitivity (not limited to episodes of mood disturbance) that results in significant social or occupational impairment
C. Criteria are not met for “with melancholic features” or “with catatonia” during the same episode.

Methods described herein, in part, treat a subject manifesting at least one or more behaviors selected from the group consisting of mood reactivity, increased sleep, hypersomnia, leaden paralysis, and long-standing pattern of interpersonal rejection sensitivity resulting in significant social or occupational impairment, each of which are terms well known to one skilled in the art. Such behaviors may be referred to as atypical depression symptoms. In embodiments, the subject further manifests one or more behaviors selected from the group consisting of depressed mood—indicated by subjective report or observation by others (in children and adolescents, can be irritable mood), loss of interest or pleasure in almost all activities—indicated by subjective report or observation by others, significant (more than 5 percent in a month) unintentional weight loss or decrease in appetite (in children, failure to make expected weight gains), significant (more than 5 percent in a month) unintentional weight gain or increase in appetite (in children, failure to make expected weight gains), sleep disturbance (insomnia or hypersomnia), psychomotor changes (agitation or retardation) severe enough to be observable by others, tiredness, fatigue, or low energy, or decreased efficiency with which routine tasks are completed, a sense of worthlessness or excessive, inappropriate, or delusional guilt (not merely self-reproach or guilt about being sick), impaired ability to think, concentrate, or make decisions—indicated by subjective report or observation by others, recurrent thoughts of death (not just fear of dying), and suicidal ideation, or suicide attempts, each of which are terms well known to one skilled in the art.

### Bing Eating Disorder (BED)

Binge eating disorder (BED) is an eating disorder characterized by recurring episodes of binge eating accompanied by a sense of lack of control and often negative feelings about oneself but without intervening periods of compensatory behavior (as self-induced vomiting, purging by laxatives, fasting, or prolonged exercise. The following criteria are required to make the diagnosis of binge eating disorder according to DSM-5:
A. Recurrent episodes of binge eating. An episode of binge eating is characterized by both of the following:
   1. Eating, in a discrete period of time (e.g., within any 2-hour period), an amount of food that is definitely larger than most people would eat in a similar period of time under similar circumstances
   2. A sense of lack of control overeating during the episode (e.g., a feeling that one cannot stop eating or control what or how much one is eating)
B. The binge-eating episodes are associated with three (or more) of the following:
   1. Eating much more rapidly than normal
   2. Eating until feeling uncomfortably full
   3. Eating large amounts of food when not feeling physically hungry
   4. Eating alone because of feeling embarrassed by how much one is eating
   5. Feeling disgusted with oneself, depressed, or very guilty after overeating
C. Marked distress regarding binge eating is present
D. The binge eating occurs, on average, at least once a week for 3 months.
E. The binge eating is not associated with the regular use of inappropriate compensatory behaviors (e.g., purging, fasting, excessive exercise) and does not occur exclusively during the course of anorexia nervosa or bulimia nervosa.
   Specify if:
   In partial remission: After full criteria for binge-eating disorder were previously met, binge eating occurs at an average frequency of less than one episode per week for a sustained period of time.
   In full remission: After full criteria for binge-eating disorder were previously met, none of the criteria have been met for a sustained period of time.
   Specify Current Severity:
   Severity is also noted in the diagnosis, from mild to extreme: Mild: 1-3 binge-eating episodes per week Moderate: 4-7 binge-eating episodes per week Severe: 8-13 binge-eating episodes per week Extreme: 14 or more binge-eating episodes per week.

### Bulimia Nervosa

Bulimia nervosa also known as simply bulimia, is an eating disorder characterized by binge eating followed by purging, and excessive concern with body shape and weight. The following criteria are required to make the diagnosis of bulimia nervosa according to DSM-5:
A. Recurrent episodes of binge eating, as characterized by both:
   1. Eating, within any 2-hour period, an amount of food that is definitively larger than what most individuals would eat in a similar period of time under similar circumstances.
   2. A feeling that one cannot stop eating or control what or how much one is eating.
B. Recurrent inappropriate compensatory behaviors in order to prevent weight gain such as self-induced vomiting; misuse of laxatives, diuretics, or other medications; fasting or excessive exercise.
C. The binge eating and inappropriate compensatory behaviors occur, on average, at least once a week for 3 months.
D. Self-evaluation is unjustifiability influenced by body shape and weight.
E. The disturbance does not occur exclusively during episodes of anorexia nervosa.
   Specify if:
   Partial remission: After full criteria were previously met, some but not all of the criteria have been met for a sustained period of time.
   Full remission: After full criteria were previously met, none of the criteria have been met for a sustained period of time.
   Current severity (the level of severity may be increased to reflect other symptoms and the degree of functional disability):
   Mild: An average of 1-3 episodes of inappropriate compensatory behaviors per week.
   Moderate: An average of 4-7 episodes of inappropriate compensatory behaviors per week. Severe: An average of 8-13 episodes of inappropriate compensatory behaviors per week. Extreme: An average of 14 or more episodes of inappropriate compensatory behaviors per week.

### Anorexia Nervosa

Anorexia nervosa is an eating disorder that causes a person to restrict their food intake. They might try to avoid eating altogether, eat very small portions, and/or cut out certain foods and eat only a select few. A common feature of anorexia is an extreme fear of being overweight (even if they are underweight). The following criteria are required to make the diagnosis of anorexia nervosa according to DSM-5:
1. Restriction of energy intake relative to requirements leading to a significantly low body weight in the context of age, sex, developmental trajectory, and physical health.
2. Intense fear of gaining weight or becoming fat, even though underweight.
3. Disturbance in the way in which one's body weight or shape is experienced, undue influence of body weight or shape on self-evaluation, or denial of the seriousness of the current low body weight.

Symptoms associated with anorexia may include:
Being underweight (sometimes severely)
Bingeing and purging (by vomiting or taking laxatives)
Constipation, bloating, and stomach pains
Dehydration
Distorted body image
Dizzy spells and faintness
Extreme tiredness (fatigue)
Extremely restrictive eating
Intense fear of gaining weight
Loss of periods or failure to begin a menstrual cycle
Loss or fluctuation of body fat and muscle
Poor circulation (constantly feeling cold)
Skin that is yellowed, dry, or covered in soft hair (lanugo)
Taking diet pills or aids
Talking about weight or food all the time
Suicidal thoughts or actions

### Subtypes of Anorexia Include:

Restricting type: During the last 3 months, the individual has not engaged in recurrent episodes of binge eating or purging behaviour (i.e., self-induced vomiting or the misuse of laxatives, diuretics, or enemas). This subtype describes presentations in which weight loss is accomplished primarily through dieting, fasting, and/or excessive exercise.

Binge-eating/purging type: During the last 3 months, the individual has engaged in recurrent episodes of binge eating or purging behaviour (i.e. self-induced vomiting or the misuse of laxatives, diuretics, or enemas).

Level of severity may include: Mild (BMI>17 kg/m²), Moderate (BMI=16-16.99 kg/m²), Severe (BMI=15-15.99 kg/m²), Extreme (BMI<15 kg/m²).

### Compound

### Compound 1

Compound 1, as depicted below, is a triple reuptake inhibitor, also known as (3,4-dichloro-phenyl)-((S)-3-propyl-pyrrolidin-3-yl)-methanone.

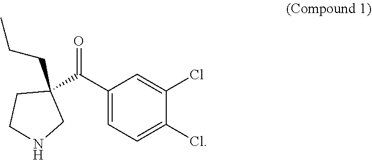 [SMILES: CCC[C@]1(C(=O)c2ccc(Cl)c(Cl)c2)CCNC1]

A method of chemically synthesizing Compound 1 is described in U.S. Pat. Nos. 8,084,623 and 9,527,810 which are incorporated by reference in their entirety.

In various embodiments, methods described herein comprise administering Compound 1 or a pharmaceutically acceptable salt thereof. In various embodiments, the pharmaceutically acceptable salt of Compound 1 can be a salt of Compound 1 with physiologically compatible mineral acids, such as hydrochloric acid, sulfuric acid, sulfurous acid or phosphoric acid; or with organic acids, such as methanesulfonic acid, p-toluenesulfonic acid, acetic acid, lactic acid, trifluoroacetic acid, citric acid, fumaric acid, maleic acid, tartaric acid, succinic acid or salicylic acid.

In certain embodiments, the pharmaceutically acceptable salt of Compound 1 is a hydrochloride salt, being in a hydrate or an anhydrate form (e.g., anhydrate, hemihydrate, monohydrate, or quarterhydrate). In certain embodiments, the pharmaceutically acceptable salt of Compound 1 is a hydrochloride salt, being in a quarterhydrate form.

In various embodiments, the pharmaceutically acceptable salt of Compound 1 is

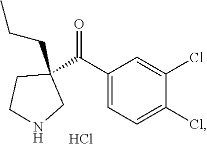 [SMILES: CCC[C@]1(C(=O)c2ccc(Cl)c(Cl)c2)CCNC1 ; Cl]

or a hydrate thereof.

In certain embodiments, Compound 1 or a pharmaceutically acceptable salt thereof is in an amorphous form. In certain embodiments, Compound 1 or a pharmaceutically acceptable salt thereof is in a crystalline form. In certain embodiments, the crystalline form is a crystalline polymorph or a hydrate thereof. In some embodiments, the crystalline polymorph is (3,4-dichloro-phenyl)-((S)-3-propyl-pyrrolidin-3-yl)-methanone hydrochloride quarterhydrate (Form 1). In some embodiments, the crystalline polymorph is (3,4-dichloro-phenyl)-((S)-3-propyl-pyrrolidin-3-yl)-methanone hydrochloride (Form 2).

### Form 1

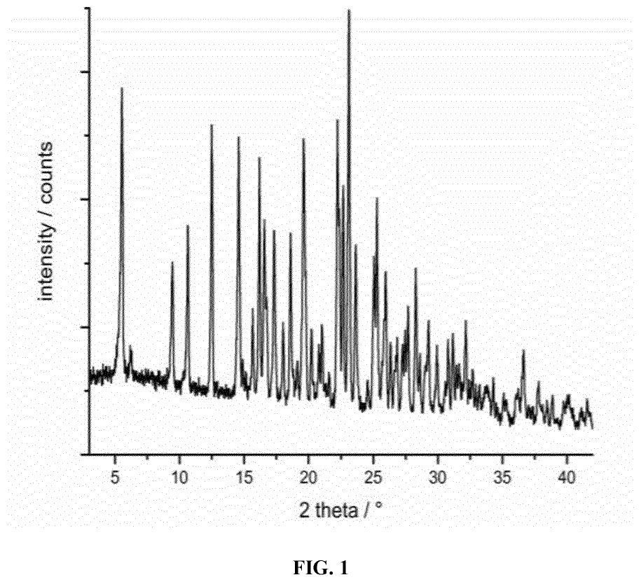

In some embodiments, the compound is in a crystalline quarterhydrate form (Form 1) of a hydrochloride salt of Compound 1, wherein Form 1 has an X-ray powder diffraction (XRPD) pattern as substantially shown in FIG. 1. In some embodiments, Form 1 is characterized by at least three peaks selected from the following X-ray powder diffraction peaks obtained with a Cu_{Kα} radiation at 2θ (2 Theta): 5.5±0.20°, 9.4±0.20°, 10.6±0.20°, 12.5±0.20°, 14.6±0.20°, 16.2±0.20°, 16.6±0.20°, 17.3±0.20°, 18.6±0.20°, 19.6±0.20°, 22.2±0.20°, 22.7±0.20°, 23.1±0.20°, 23.7±0.20° and 25.3±0.20°.

### Form 2

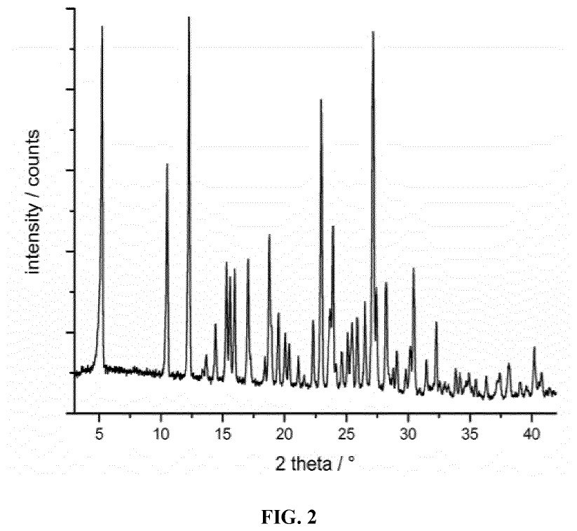

In some embodiments, the compound is in a crystalline form (Form 2) of a hydrochloride salt of Compound 1, wherein Form 2 has an X-ray powder diffraction (XRPD) pattern as substantially shown in FIG. 2. In some embodiments, Form 2 is characterized by at least three peaks selected from the following X-ray powder diffraction peaks obtained with a Cu_{Kα} radiation at 20 (2 Theta): 5.2±0.20°, 10.5±0.20°, 12.3±0.20°, 15.3±0.20°, 15.6±0.20°, 16.0±0.20°, 17.1±0.20°, 18.8±0.20°, 23.0±0 0.20°, 23.9±0.20°, 27.2±0.20°, 28.2±0.20° and 30.5±0.20°.

In one aspect, the present disclosure relates to a composition such as a pharmaceutical composition comprising Compound 1, or a pharmaceutically acceptable salt thereof, and a pharmaceutically acceptable excipient, for the treatment of atypical depression in a subject in need thereof. In another aspect, the present disclosure relates to a composition such as a pharmaceutical composition comprising Compound 1, or a pharmaceutically acceptable salt thereof, and a pharmaceutically acceptable excipient, for the treatment of a medical condition associated with atypical depression in a subject in need thereof. In various embodiments, the composition is a solid pharmaceutical composition.

In various embodiments, the amount of Compound 1, or a pharmaceutically acceptable salt thereof, in the pharmaceutical compositions described herein can be from about 1 mg to about 70 mg of Compound 1, or a pharmaceutically acceptable salt thereof. In some embodiments, the composition comprises from about 1 mg to about 60 mg. In some embodiments, the composition comprises from about 1 mg to about 45 mg. In some embodiments, the composition comprises from about 1 mg to about 30 mg. In some embodiments, the composition comprises from about 1 mg to about 15 mg. In some embodiments, the composition comprises from about 1 mg to about 5 mg. In some embodiments, the composition comprises about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, or about 70 mg.

In various embodiments, the amount of Compound 1, or a pharmaceutically acceptable salt thereof, in the pharmaceutical compositions described herein can be from about 1 mg/kg to about 70 mg/kg of Compound 1, or a pharmaceutically acceptable salt thereof. In some embodiments, the composition comprises from about 1 mg/kg to about 60 mg/kg. In some embodiments, the composition comprises from about 1 mg/kg to about 45 mg/kg. In some embodiments, the composition comprises from about 1 mg/kg to about 30 mg/kg. In some embodiments, the composition comprises from about 1 mg/kg to about 15 mg/kg. In some embodiments, the composition comprises from about 1 mg/kg to about 5 mg/kg. In some embodiments, the composition comprises about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg.

In various embodiments, the pharmaceutical compositions described herein comprise a therapeutically effective amount of the free base form of Compound 1.

In various embodiments, the pharmaceutical compositions described herein comprise a therapeutically effective amount of a pharmaceutically acceptable salt of Compound 1. In some embodiments, the pharmaceutically acceptable salt of Compound 1 can be a salt of Compound 1 with physiologically compatible mineral acids, such as hydrochloric acid, sulfuric acid, sulfurous acid or phosphoric acid; or with organic acids, such as methanesulfonic acid, p-toluenesulfonic acid, acetic acid, lactic acid, trifluoroacetic acid, citric acid, fumaric acid, maleic acid, tartaric acid, succinic acid or salicylic acid.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In certain embodiments, the pharmaceutical compositions disclosed herein are administered orally.

The pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions provided herein may be presented in unit dosage forms to facilitate accurate dosing. The term “unit dose” or “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. In various embodiments, the pharmaceutical dosage forms described herein can be administered as a unit dose. Typical unit dosage forms include prefilled, premeasured ampules or syringes of the liquid compositions or pills, tablets, capsules or the like in the case of solid compositions.

The pharmaceutical compositions provided herein may be presented in sustained release form. A sustained release form is a formulation which is designed to slowly release a therapeutic agent in the body over an extended period of time. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

In various embodiments, the pharmaceutical compositions provided herein are administered to the patient as a solid dosage form. In certain embodiments, the solid dosage form is a capsule. In certain embodiments, the solid dosage form is a tablet.

In various embodiments, the pharmaceutical compositions provided herein comprise Compound 1 as the sole active agent, or in combination with other active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from the group consisting of two or more of the recited elements or components.

Further, it should be understood that elements and/or features of a composition or a method described herein can be combined in a variety of ways without departing from the spirit and scope of the present invention, whether explicit or implicit herein. For example, where a reference is made to a particular compound, that compound can be used in various embodiments of methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., at least one) of the grammatical object of the article, unless the context is inappropriate. By way of example, “an element” means one element or more than one element.

The term “and/or” is used in this disclosure to mean either “and” or “or” unless indicated otherwise.

The expression “and/or” in connection with three or more recited objects should be understood to have the same meaning unless otherwise understood from the context.

The use of the term “comprise,” “comprises,” “comprising,” “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “composition” or “pharmaceutical composition” or “pharmaceutical formulation” refers to the combination of an active agent with an excipient or a carrier, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or a human, as appropriate, and/or that are approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., Compound 1), which salt is compatible with pharmaceutical administration.

Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, monosulfate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration, transmucosal administration (e.g., buccal, sublingual, nasal, or transdermal), or subcutaneous administration, or the implantation of a slow-release device, e.g., a mini-osmotic pump, to a subject. Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc.

The term “persist” as used herein refers to a behavior being persistent in a subject, in which the behavior is persistently present in the subject for an extended period of time or for a predetermined time. In some embodiments, the behavior persists in a subject at least 3, 6, or 12 hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days in the subject. In some embodiments, the behavior persists in a subject at least 1, 2, 3, 4, 5, 6, 7, 8, 9, 10, 11, or 12 months in the subject.

As used herein, and unless otherwise specified, the terms “treat,” “treating” and “treatment” include an action that occurs while a subject is suffering from the specified disease, disorder or condition, which reduces the severity of the disease, disorder or condition, or retards or slows the progression of the disease, disorder or condition (e.g., “therapeutic treatment”). “Treat,” “treating” and “treatment”, as used herein, can include any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The phrase “therapeutically effective amount,” as used herein, refers to the amount of a compound (e.g., Compound 1), or a pharmaceutically acceptable salt thereof, that will elicit the biological or medical response of a tissue, system, animal or human that is being sought by the researcher, veterinarian, medical doctor or other clinician. The compound, or a pharmaceutically acceptable salt thereof, described in the present disclosure can be administered in therapeutically effective amounts to treat a disease. A therapeutically effective amount of a compound, or a pharmaceutically acceptable salt thereof, can be the quantity required to achieve a desired therapeutic and/or prophylactic effect, such as an amount which results in lessening of a symptom of a disorder such as atypical depression.

As used herein, “an impulsive behavior” refers to behavior that is characterized by a significant lack of impulse control followed by guilt feelings. Impulsive behaviors include mood reactivity, increased sleep, hypersomnia, leaden paralysis, long-standing pattern of interpersonal rejection sensitivity resulting in significant social or occupational impairment, binge eating not associated with the regular use of inappropriate compensatory behaviors, binge eating not occurring exclusively during the course of anorexia nervosa or bulimia nervosa, recurrent inappropriate compensatory behaviors, such as self-induced vomiting; misuse of laxatives, diuretics, or other medications, in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior, recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, increased appetite, hyperphagia, and significant lack of impulse control followed by guilt feelings. Each behavior described herein is well known to one skilled in the art.

As used herein, “sufficient to improve at least one of the behaviors” means a subject's behavior improves when comparing behaviors before administration of Compound 1 to behaviors after administration of Compound 1, as measured or diagnosed in the Diagnostic and Statistical Manual of Mental Disorders. For example, reducing the amount of food intake by a subject in a day or within any 2-hour period, as compared to the food intake of the subject before administration of Compound 1. In another example, reducing the amount of heavy and leaden feelings in arms or legs in a subject in a day, as compared to the food intake of the subject before administration of Compound 1.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

In the Examples provided below, the following abbreviations are used: “PROG” refers to progressive ratio; “FR1” refers to fixed ration of one or fixed ratio 1; “SEM” refers to standard error of mean; “VEH” refers to vehicle; “ANOVA” refers to analysis of variance test; “NR” refers to non-rapid eye movement sleep; “REM” refers to rapid eye movement sleep; “p.o.” or “per os” refers to by mouth; “CAF” refers to caffeine; “DAT” refers to dopamine transporters; “SERT” refers to serotonin transporters; “TRI” refers to triple reuptake inhibitor; “NR” refers to non-rapid eye movement sleep; “REM” refers to rapid eye movement sleep

### Example 1. Synthesis of (3,4-dichloro-phenyl)-((S)-3-propyl-pyrrolidin-3-yl)-methanone hydrochloride (15) quarterhydrate [See U.S. Pat. No. 9,527,810]

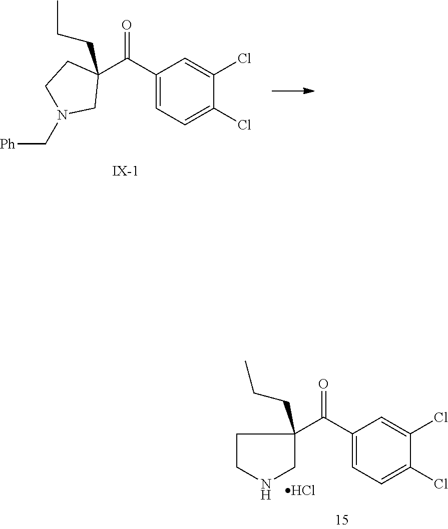

(S)-(1-benzyl-3-propylpyrrolidin-3-yl)(3,4-dichlorophenyl)methanone (IX-1) (5 g, 13.3 mmol, Eq: 1.00, see U.S. Pat. No. 9,527,810 for synthesis) was dissolved in dichloromethane (30 mL). The light yellow solution was cooled to 0-5° C. and N-ethyldiisopropylamine (172 mg, 226 μL, 1.33 mmol, Eq: 0.1) was added. 1-Chloroethyl chloroformate (2.28 g, 1.74 ml, 15.9 mmol, Eq: 1.2) was added dropwise while the temperature was maintained in between 0-5° C. The reaction was warmed to room temperature over 30 min and was stirred 1 h at room temperature. Methanol (25 mL) was added and the light yellow solution was heated to 40° C. for 40 min. The reaction mixture was concentrated under reduced pressure (40° C., 600-15 mbar) to give 5.48 g of crude product. Ethyl acetate (30.0 mL) was added and the suspension was heated to 50° C. A solution of water (239 mg, 239 μL, 13.3 mmol, Eq: 1.0) in ethyl acetate (35 mL) was added over 10 min. The white suspension was stirred for 1 h at 50° C. and cooled to room temperature over 1.5 h. The suspension was filtered, and the filter cake was washed twice with ethyl acetate (10 mL) and dried under reduced pressure (400 C, 15 mbar) to give 4.02 g of (15) as quarterhydrate (93% yield).

### Example 2. Synthesis of (3,4-dichloro-phenyl)-((S)-3-propyl-pyrrolidin-3-yl)-methanone hydrochloride (15) quarterhydrate [See U.S. Pat. No. 9,527,810]

### 1. Grignard Formation (A′)

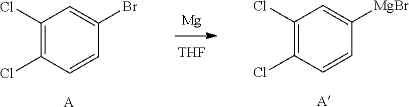

6.8 g of Magnesium (279 mmol, 1.3 equiv.) was suspended in 60 mL tetrahydrofuran. The suspension was heated to 40° C., and 2% of a solution of 70.5 g 3,4-dichlorobromobenzene (A) in 200 mL tetrahydrofuran was added (the Grignard started within a few minutes). After the exotherm ceased, the remaining aryl bromide solution was added over 2 h. The reaction mixture was stirred for 1 h at 40° C., and then the mixture was cooled to room temperature.

### 2. Grignard Addition

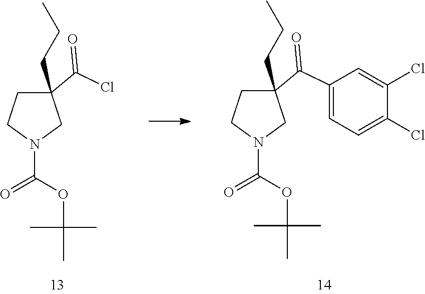 [SMILES: CCC[C@]1(C(=O)Cl)CCN(C(=O)OC(C)(C)C)C1 ; CCC[C@]1(C(=O)c2ccc(Cl)c(Cl)c2)CCN(C(=O)OC(C)(C)C)C1]

A solution of the acid chloride (13) (see U.S. Pat. No. 9,527,810 for synthesis) was degassed 3 times and 55.8 g of N,N,N′,N′,N″-pentamethyldiethylenetriamine (PMDTA) (322 mmol, 1.5 equiv.) was added. The light suspension was heated to 40-45° C., and the Grignard solution (A′) was added dropwise over 1.5 h. After 1 h of additional reaction time, the reaction mixture was cooled to room temperature. 500 mL of 2 M HCl (aq), 300 mL saturated NaCl (aq) and 300 mL ethanol were added. The organic phase was separated and washed with a mixture consisting of 500 mL 2 M HCl (aq), 300 mL saturated NaCl (aq) and 300 mL ethanol. The organic phase was washed with 400 mL of 1M NaOH (aq) and twice with 150 mL 10% NaCl (aq). The organic phase was concentrated under reduced pressure to an oil, taken up in 100 mL toluene and concentrated again to give 87 g of crude (14) with 80% purity.

3. (3,4-Dichloro-phenyl)-((S)-3-propyl-pyrrolidin-3-yl)-methanone hydrochloride (15) as quarterhydrate

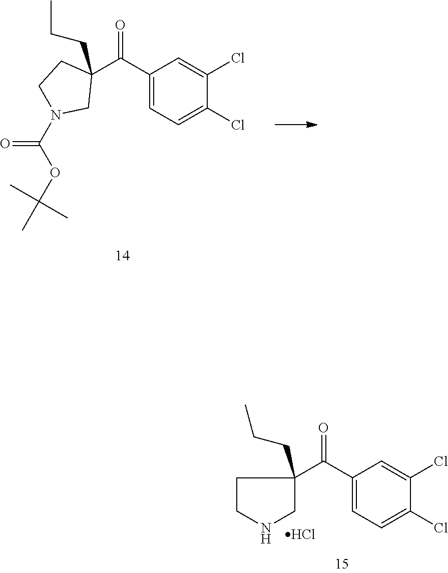 [SMILES: CCC[C@]1(C(=O)c2ccc(Cl)c(Cl)c2)CCN(C(=O)OC(C)(C)C)C1 ; "14" ; CCC[C@]1(C(=O)c2ccc(Cl)c(Cl)c2)CCNC1 ; Cl]

The crude (14) was dissolved in 130 mL of toluene and the solution was added dropwise to a mixture of 140 mL toluene and 70 mL 37% HCl (aq) at 60-70° C. After 1 h, the reaction mixture was azeotroped with toluene (Tr max=70° C., Tj max=120° C., reduced pressure) and adjusted to a volume of 350-400 mL. A mixture consisting of 700 mL ethyl acetate and 3.6 mL water was added at 65° C. The solution was cooled to room temperature over 1 h, during which crystallization started (around 45° C.). After stirring overnight, the suspension was cooled to 0-5° C. for 2 h, filtered and washed twice with 200 mL ethyl acetate. The crystals were re-suspended in 250 mL of ethyl acetate, digested at 55° C. for 2 h, and then cooled to room temperature. The suspension was filtered, and the filter cake was washed with 200 mL ethyl acetate. The crystals were dried at 50° C. under reduced pressure to give 54.4 g of the (3,4-dichloro-phenyl)-((S)-3-propyl-pyrrolidin-3-yl)-methanone hydrochloride quarterhydrate as a powder with 99% purity.

An Alternative Route for (3,4-dichloro-phenyl)-((S)-3-propyl-pyrrolidin-3-yl)-methanone hydrochloride (15) as quarterhydrate

The crude (14) was dissolved in 63 mL toluene and deprotected at 60° C. with 24 mL 37% HCl (aq). After completion of the reaction, the reaction mixture was dried azeotropically with toluene at 50-60° C. The solution was cooled to room temperature, and 100 mL water was added. The aqueous phase was separated and washed with 50 mL toluene. The aqueous phase was dried azeotropically with toluene and concentrated to dryness to give 22.5 g of (3,4-dichloro-phenyl)-((S)-3-propyl-pyrrolidin-3-yl)-methanone hydrochloride quarterhydrate (90% yield). (3,4-Dichloro-phenyl)-((S)-3-propyl-pyrrolidin-3-yl)-methanone hydrochloride quarterhydrate can be obtained by digestion or recrystallization, for example, by processes described below.

Transformation of (3,4-dichloro-phenyl)-((S)-3-propyl-pyrrolidin-3-yl)-methanone hydrochloride anhydrate to the quarterhydrate form:

(3,4-Dichloro-phenyl)-((S)-3-propyl-pyrrolidin-3-yl)-methanone hydrochloride anhydrate (40 g, 124 mmol, Eq: 1.00, see Example 3 or U.S. Pat. No. 9,527,810 for synthesis) was suspended in a mixture of ethyl acetate (340 mL), ethanol (36 mL) and water (0.6 mL) at room temperature. The suspension was heated to 40° C. and a mixture consisting of ethyl acetate (20 mL), ethanol (0.5 mL) and water (0.6 mL) was added over 1 h. The suspension was cooled to room temperature over 1 h. After stirring overnight at room temperature, the suspension was cooled over 2-3 h at 0-5° C., filtered and washed with a cold (0-5° C.) mixture of ethyl acetate (55 mL), ethanol (5 mL) and water (0.5 mL). The filter cake was dried at 50° C. under reduced pressure to give 38 g of (3,4-dichloro-phenyl)-((S)-3-propyl-pyrrolidin-3-yl)-methanone hydrochloride quarterhydrate (1.5% water).

Recrystallization of (3,4-dichloro-phenyl)-((S)-3-propyl-pyrrolidin-3-yl)-methanone hydrochloride quarterhydrate:

54.4 g of (3,4-dichloro-phenyl)-((S)-3-propyl-pyrrolidin-3-yl)-methanone hydrochloride quarterhydrate was dissolved at room temperature in 550 mL ethanol. The solution was filtered and concentrated under reduce pressure at 60° C. to a volume of 140 mL. The volume was adjusted to 550 mL by the addition of ethyl acetate. The remaining ethanol was solvent exchanged to ethyl acetate (60° C., reduced pressure), and 55 mL ethanol was added to the resulting suspension at 60° C. 1.5 mL water was then added and the solution was slowly cooled to room temperature, during which the crystallization occurred. After stirring at room temperature overnight, the suspension was cooled to 0-5° C. for 1 h and was filtered. The filter cake was washed with a mixture of 50 mL ethyl acetate and 5 mL ethanol, followed by two more washes with 50 mL ethyl acetate. The crystals were dried at 50° C. overnight under reduced pressure to give 48.9 g of (3,4-dichloro-phenyl)-((S)-3-propyl-pyrrolidin-3-yl)-methanone hydrochloride quarterhydrate as a powder with 99% purity.

### Example 3. Synthesis of (3,4-dichloro-phenyl)-((S)-3-propyl-pyrrolidin-3-yl)-methanone hydrochloride (15) anhydrate [See U.S. Pat. No. 9,527,810]

50 g of Sodium salt was transformed into the corresponding acid chloride (13) (see U.S. Pat. No. 9,527,810 for synthesis). The acid chloride (13) was reacted with 3,4-dichlorophenyl-MgBr (A′), and then deprotected. After azeotrope drying, an orange turbid toluene solution (300 g, water content <0.1%) was obtained by Karl Fischer titration of the crude (15).

(i) ⅕ of the crude (15) solution (max theoretical content: 11.3 g of (15)) was cooled to room temperature. After storing for overnight at room temperature, the resulting suspension was filtered. The filter cake was washed with ethyl acetate (Karl Fischer titration of wet filter cake 0.2%) and dried at 50-60° C. under reduced pressure to give 5.9 g of (15) crystals.

(ii) ⅕ of the crude (15) solution (max theoretical content: 11.3 g of (15)) was cooled to room temperature. After storing for 4 days at room temperature, the resulting suspension was stirred at 0-2° C. for 4 h. The resulting suspenction was filtered, the filter cake was washed with ethylacetate (Karl Fischer titration of wet filter cake 0.2%) and dried at 50-60° C. under reduced pressure to give 8.8 g of (15) crystals.

### Example 4. Crystalline Forms of (3,4-dichloro-phenyl)-((S)-3-propyl-pyrrolidin-3-yl)-methanone hydrochloride and (3,4-dichloro-phenyl)-((S)-3-propyl-pyrrolidin-3-yl)-methanone hydrochloride quarterhydrate [See U.S. Pat. No. 9,527,810]

Characterization methods and data of crystalline forms of Compound 1 are described in U.S. Pat. No. 9,527,810, which is incorporated by reference in its entirety.

### X-ray Powder Diffraction (XRPD)

X-ray diffraction powder patterns were recorded at ambient conditions in transmission geometry with a STOE STADI P diffractometer (Cu_{Kα} radiation, primary monochromator, position sensitive detector, angular range 3° to 420 (2 Theta), approximately 60 minutes total measurement time). The samples were prepared and analyzed without further processing (e.g. grinding or sieving) of the substance.

XRPD Pattern of (3,4-dichloro-phenyl)-((S)-3-propyl-pyrrolidin-3-yl)-methanone hydrochloride quarterhydrate (Form 1)

The hydrochloride quarterhydrate of (3,4-dichloro-phenyl)-((S)-3-propyl-pyrrolidin-3-yl)-methanone solid (Form 1) can be identified by as few as one characteristic peak in its powder X-ray diffraction patterns as shown in FIG. 1. Exemplary X-ray powder diffraction patterns of hydrochloride quarterhydrate of (3,4-dichloro-phenyl)-((S)-3-propyl-pyrrolidin-3-yl)-methanone (Form 1) in terms of 20 (2 Theta) are: 5.5±0.20°, 9.4±0.20°, 10.6±0.20°, 12.5±0.20°, 14.6±0.20°, 16.2±0.20°, 16.6±0.20°, 17.3±0.20°, 18.6±0.20°, 19.6±0.20°, 22.2±0.20°, 22.7±0.20°, 23.1±0.20°, 23.7±0.20° and 25.3±0.20°; in particular characteristic peaks are 9.4±0.20°, 14.6±0.20°, 16.6±0.20°, 19.6±0.20° and 22.2±0.20°.

XRPD Pattern of (3,4-dichloro-phenyl)-((S)-3-propyl-pyrrolidin-3-yl)-methanone hydrochloride (Form 2)

The hydrochloride of (3,4-dichloro-phenyl)-((S)-3-propyl-pyrrolidin-3-yl)-methanone solid (Form 2) can be identified by as few as one characteristic peak in its powder X-ray diffraction patterns as shown in FIG. 2. Exemplary X-ray powder diffraction patterns of hydrochloride of (3,4-dichloro-phenyl)-((S)-3-propyl-pyrrolidin-3-yl)-methanone solid (Form 2) in terms of 20 (2 Theta) are: 5.2±0.20°, 10.5±0.20°, 12.3±0.20°, 15.3±0.20°, 15.6±0.20°, 16.0±0 0.20°, 17.1±0.20°, 18.8±0.20°, 23.0±0.20°, 23.9±0.20°, 27.2±0.20°, 28.2±0.20° and 30.5±0.20°.

### Crystal Structure Analysis

For single crystal structure analysis, a single crystal was mounted in a loop on a goniometer and measured at ambient conditions. Data were collected on a GEMINI R Ultra diffractometer from Oxford Diffraction (Oxford). Cu-radiation of 1.54 Å wavelength was used for data collection. Data was processed with the software CRYSALIS. The crystal structure was solved and refined with standard crystallographic software. In this case, the program ShelXTL from Bruker AXS (Karlsruhe) was used.

Preparation of Single Crystals of (3,4-dichloro-phenyl)-((S)-3-propyl-pyrrolidin-3-yl)-methanone monohydrochloride quarterhydrate

10 mg of (3,4-dichloro-phenyl)-((S)-3-propyl-pyrrolidin-3-yl)-methanone hydrochloride were dissolved in 0.226 mL of nitromethane at 60° C. The solution was allowed to reach the ambient temperature without agitation. After 24 h, single crystals were harvested and subjected to X-ray crystal structure analysis.

Structural data derived from (3,4-dichloro-phenyl)-((S)-3-propyl-pyrrolidin-3-yl)-methanone hydrochloride quarterhydrate single crystal X-ray analysis are the following unit cell parameters:

| | | |
|---|---|---|
| a | 6.14 | Å |
| b | 16.70 | Å |
| c | 17.43 | Å |

| | |
|---|---|
| alpha | 66.73° |
| beta | 81.47° |
| gamma | 86.51° |

wherein a, b and c are each a representative length of the crystal lattice, and alpha, beta and gamma are unit cell angles. The salt crystallizes in the space group P1, affording a cell volume of 1623.82 Å³.

### Differential Scanning Calorimetry (DSC)

Differential Scanning Calorimetry curves were recorded using a Mettler-Toledo™ differential scanning calorimeter DSC820, DSC821 or DSC1 with a FRS05 sensor. System suitability tests were performed with Indium as reference substance and calibrations were carried out using Indium, Benzoic acid, Biphenyl and Zinc as reference substances.

For the measurements, approximately 2-6 mg of sample were placed in aluminum pans, accurately weighed and hermetically closed with perforation lids. Prior to measurement, the lids were automatically pierced resulting in approximately 1.5 mm pin holes. The samples were then heated under a flow of nitrogen of about 100 mL/min using heating rates of usually 10 K/min.

### Thermal Gravimetric Analysis (TGA)

Thermal Gravimetric Analysis was performed on a Mettler-Toledo™ thermogravimetric analyzer (TGA850 or TGA851). System suitability tests were performed with Hydranal as reference substance and calibrations using Aluminum and Indium as reference substances.

For the thermogravimetric analyses, approximately 5-10 mg of sample were placed in aluminum pans, accurately weighed and hermetically closed with perforation lids. Prior to the measurement, the lids were automatically pierced resulting in approximately 1.5 mm pin holes. The samples were then heated under a flow of nitrogen of about 50 mL/min using a heating rate of 5 K/min.

### Example 5. Assessment of Compound 1 for Appetite Suppressant Effects Using Progressive Ratio/Chow Feeding Choice Task in Rats

A study to evaluate Compound 1 for appetite suppressant effects were conducted. The PROG/Chow feeding choice procedure was used as a test designed to evaluate the effort a rat is willing to make to eat a palatable food by pressing a lever instead of eating freely available standard food.

Methods. Thirty-one (n=31) adult male Sprague Dawley rats were used in this study. The animals were restricted to 85% of their free-feeding weight, with modest growth allowed throughout the study period. Animals were assessed using operant conditioning chambers. Animals were first introduced to the high carbohydrate pellets (“palatable food”) and trained to lever press using a continuous reinforcement schedule with a fixed ration of one (FR1=one active lever press trigger the delivery of the palatable food) for one week. After this, the rats were trained on the progressive ratio (progressively more lever pressures were needed to trigger the delivery of the palatable food) schedule alone for nine weeks at which point chow (“standard food”) was introduced (=PROG/Chow feeding choice procedure), and animals trained for another 5 weeks to acquire a steady baseline. Each session lasted 30 minutes and rats ran in the operant chambers once a day, five days a week (Monday-Friday). Vehicle (VEH), or Compound 1 doses of 5.0, 10.0, and 20.0 mg/kg were administered via oral gavage once a week (Thursday or Friday) after the training period. Treatment conditions were selected randomly. Lever presses and chow consumption (including spillage) were recorded after each operant session.

Statistical analyses. Repeated measures analysis of variance test (ANOVA) and planned comparisons were used to assess the data overall. Factorial ANOVA was used to assess group x treatment interaction. If significant interactions were found, planned comparisons (using the overall error term) were used to assess the groups individually. Dunnett's tests were also used to analyze differences between drug treatments and VEH. A p value of <0.05 was considered statistically significant for all analyses.

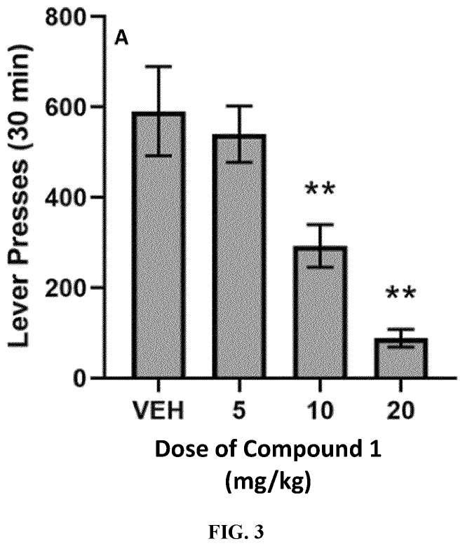

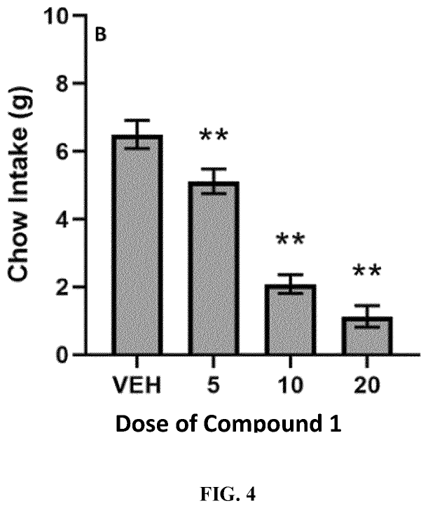

Results. All treatment conditions showed significant decreases in standard chow intake when compared to VEH (5.0 mg/kg: [F(1,90)=15.589, p<0.001], 10.0 mg/kg [F(1,90)=159.881, p<0.001], and 20.0 mg/kg: [F(1,90)=236.567, p<0.001]), as shown in FIG. 3. No significant difference between VEH and the 5.0 mg/kg dose [F(1,90)=0.391, p=n.s.], significant decreases in lever pressing were seen at both the 10.0 mg/kg dose [F(1,90)=13.534, p<0.001] and the 20.0 mg/kg dose [F(1,90)=38.407, p<0.001], as shown in FIG. 4.

The study reveals that Compound 1 demonstrates appetite suppressant effects. The pattern of decreased lever pressing and chow intake as shown in this study of PROG/Chow feeding choice task is also shown in other studies with drugs and conditions known for suppressing appetite. Examples of the drugs include fenfluramine, pre-feeding, cannabinoid antagonists and inverse agonists, and fluoxetine.

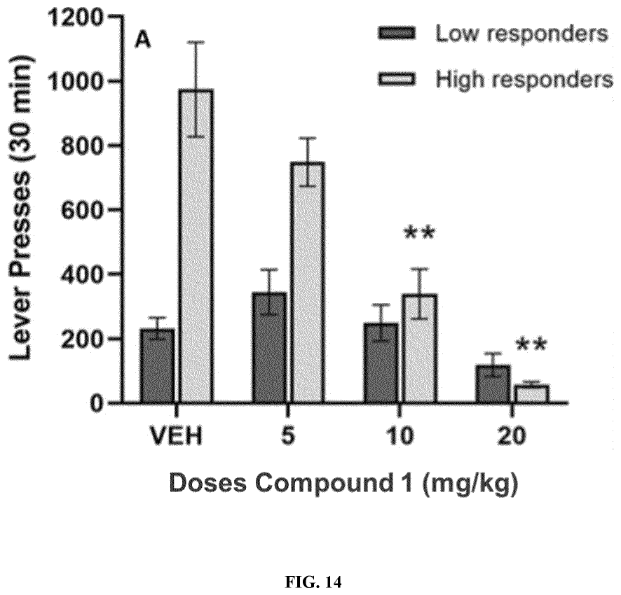

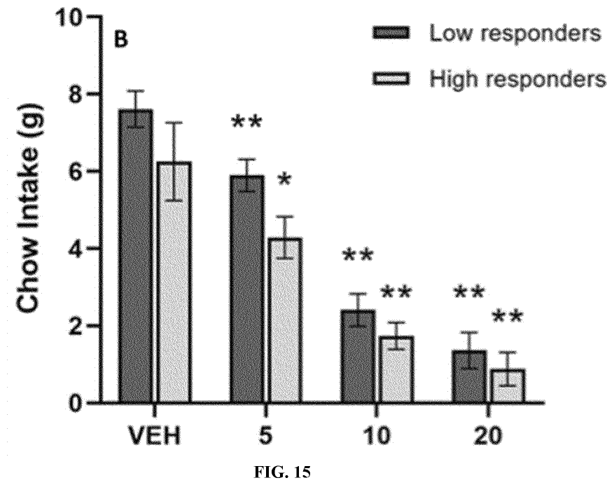

High vs. Low Responders. Factorial ANOVA on lever pressing revealed a significant group x treatment interaction [F(3,90)=13.824, p<0.001; ηp²=0.323] and a significant interaction of the linear trend (p<0.001). Due to this significant interaction, the high and low responder level pressing was analyzed separately. Significant treatment effects were found in both groups (high: p<0.001; low: p=0.01). High responder lever presses showed a significant linear trend (p<0.001). Planned comparisons revealed significant decreases in lever pressing at both the 10.0 mg/kg [F(1,42)=26.735, p<0.001] and 20.0 mg/kg [F(1,42)=55.797, p<0.001] doses when compared to VEH. There were no significant difference found when comparing the 5.0 mg/kg dose to VEH conditions [F(1,42)=3.372, p=n.s.](FIG. 14). Planned comparisons showed no significant differences when comparing any of the drug conditions to VEH (5.0 mg/kg: [F(1,45)=0.319, p=n.s.], 10.0 mg/kg: [F(1,45)=0.079, p=n.s.], 20.0 g mg/kg: [F(1,45)=3.157, p=n.s.]). However, a significant quadratic trend (p=0.01) was found in the low responder group, as shown in FIG. 14. A factorial ANOVA was also used to assess the concurrent chow Intake. A significant group×treatment interaction [F(3,90)=3.191, p<0.05; η²=0.099] was found, as well as an interaction of linear trend (p<0.05). This significant interaction allowed for separate analysis of the high and low responders. Significant treatment effects (p<0.001) and significant linear trend (p<0.001) were found in both high and low responders. In the high responder group, significant decreases in chow intake were found at the 5.0 mg/kg [F(1,42)=13.450, p<0.010], 10.0 mg/kg [F(1,42)=70.714, p<0.001], and 20.0 g mg/kg [F(1,42)=100.204, p<0.001] groups when compared to VEH through planned comparisons. Low responders also showed significant decreases in lever presses at all dose treatments (5.0 mg/kg [F(1,45)=16.976, p<0.001], 10.0 mg/kg [F(1,45)=156.918, p<0.001], 20.0 mg/kg [F(1,45)=226.122, p<0.001]) as determined by Dunnett's test (FIG. 15).

### Example 6. Assessment of Compound 1 for Appetite Suppressant Effects Using a Fixed Ratio Schedule in Rats

Another study to evaluate Compound 1 for appetite suppressant effects were conducted. A fixed ratio 1 (FR1) schedule was used, and this task is the most food dense and appetite dependent operant schedule, with high sensitivity to appetite-related manipulations such as pre-feeding and appetite suppressant drugs.

Methods. The same adult male Sprague Dawley rats (n=31) as in Example 5 were used in this study. Animals trained once daily on the PROG/Chow feeding choice procedure from Monday through Thursday. On Friday, the testing day, the animals were switched from the PROG/Chow feeding choice procedure to FR1 with no prior training. Each operant session lasted 30 minutes and lever presses were recorded at the end of the run. Drug treatment and the FR1 probe occurred on the Friday after two weeks of washout period from Example 5. A dose of 20.0 mg/kg of Compound 1 or vehicle was administered via oral gavage on the testing day. Only the highest dose was tested in this study because, in Example 5, it produced the most robust decrease in lever pressing and chow intake compared to VEH. Treatment conditions were assigned randomly. Treatment group difference was assessed by unpaired t-test. A p value of <0.05 was considered statistically significant.

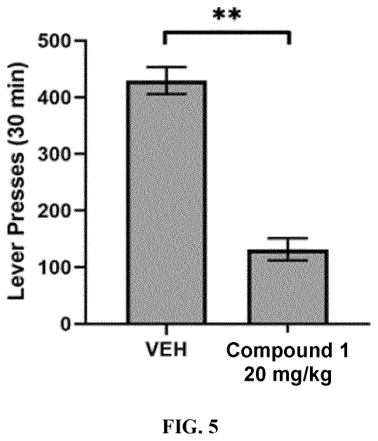

Results. Compound 1 caused a significant decrease in lever pressing in the FR1 probe study, as measured by an unpaired t-test (t=9.74; p<0.001). As shown in FIG. 5, there is a significant decrease at the 20.0 mg/kg dose when compared to the vehicle (VEH). Importantly, 6 of the 16 rats that received the high drug dose pressed the lever but refrained from eating some of the pellets. After the completion of the 30-minute operant session, approximately 5-10 uneaten pellets were left in the food dishes of these animals which is an additional marker of appetite suppression. Accordingly, together with the results in the study of Example 5, the study highlights that Compound 1 demonstrates appetite suppresant effects.

### Example 7. Assessment of Compound 1 for Appetite Suppressant Effects Using Binge-Like Eating of Chocolate

Another study to evaluate Compound 1 for appetite suppressant effects was conducted, on binge-like eating of chocolate.

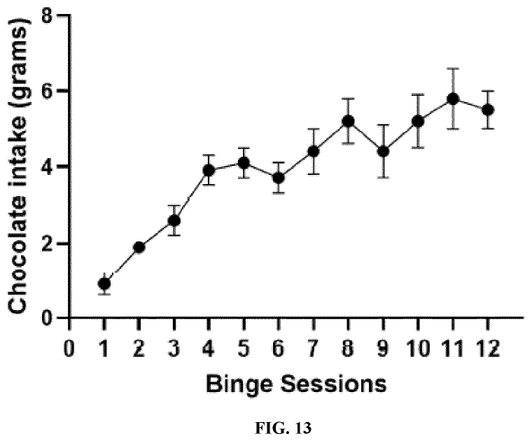

Methods. A new group of male Sprague-Dawley rats (n=8; initial weight 300-325 g) was used for the binge-like eating experiment. They were pair-housed under the same conditions described above, however, they were not food restricted, and in the home cage were allowed ad libitum access to food (laboratory chow) and water. Acquisition of the binge-like eating took place over the course of 12 exposure sessions. A 3-day, 1-hr habituation exposure to an empty feeding cage with a ceramic dish took place before chocolate exposure. With chocolate exposure, rats received chocolate (0.3 g fat, 0.57 g carbohydrate, and 0.073 g protein with a total of 5.34 kcal/g) on days 1, 2, 4, 6, 7, 9, 12, 14, 15, 18, 23, and 28. On exposure days, rats were placed in an empty feeding cage with a ceramic dish containing ground chocolate for 1 hr. Weight of chocolate was taken before and after each session to determine intake. These acquisition procedures led to gradually increased levels of chocolate intake across sessions (from 0.9±0.3 g chocolate on the first session to 5.5±0.5 g on the 12^{th} session (see FIG. 13)

Drug Administration. Upon completion of the 12 acquisition sessions of binge-like eating rats continued to be exposed to chocolate sessions twice a week for 1-hr in empty feeding cages with a ceramic dish containing chocolate. One session was for maintenance of the binge-like behavior, and then the second was for drug treatment. These sessions took place randomly throughout the week with the maintenance session always occurring prior to the drug treatment session. Compound 1 was dissolved in 0.3% Tween 80 in dH₂O. Vehicle (VEH), or doses of 5.0, 10.0, and 20.0 mg/kg were administered via oral gavage once a week for testing. A pre-treatment lead time of four hours was used. A within-subjects design was used in this study, with each rat receiving one treatment per week for a total of four weeks. Treatment conditions were selected randomly.

Data Analysis. Repeated measures ANOVA and planned comparisons were used to assess the effect of Compound 1 on binge-like eating of chocolate.

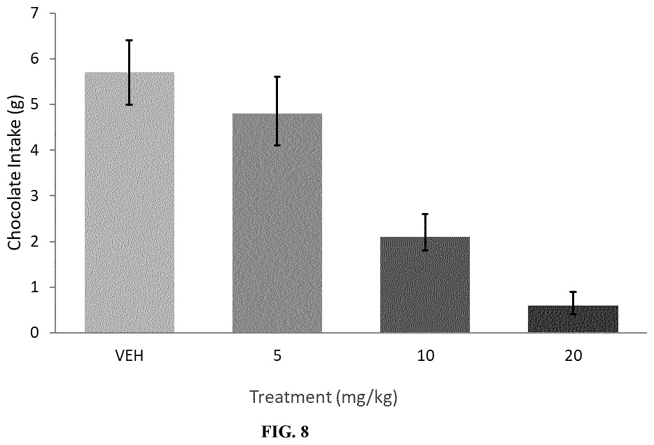

Results. The results of the binge-like eating experiment are shown in FIG. 8, FIG. 9, and FIG. 13. FIG. 13 depicts the acquisition of binge-like chocolate intake over the 12 training sessions. FIG. 8 shows the suppressive effects of Compound 1 administration of binge-like eating. There was an overall significant effect of Compound 1 treatment [F(3,21)=17.3, p<0.001]. Planned comparisons revealed that the 5.0 mg/kg dose of Compound 1 did not significantly suppress chocolate intake [F(1,21)=1.5, n.s.], but there were significant reductions in chocolate intake at the 10.0 [F (1,21)=20.4, p<0.001] and 20.0 [F(1,21)=4.09, p<0.001] mg/kg doses.

### Example 8. Assessment of Compound 1 on Sleep Using Repeated Measures in Rats

Using a counter-balanced, repeated measures design, three doses of Compound 1 (1-10 mg/kg, p.o.) were tested for their effects on sleep/wake parameters in adult male Sprague-Dawley rats (n=8). The results were compared with the effects on caffeine (10 mg/kg).

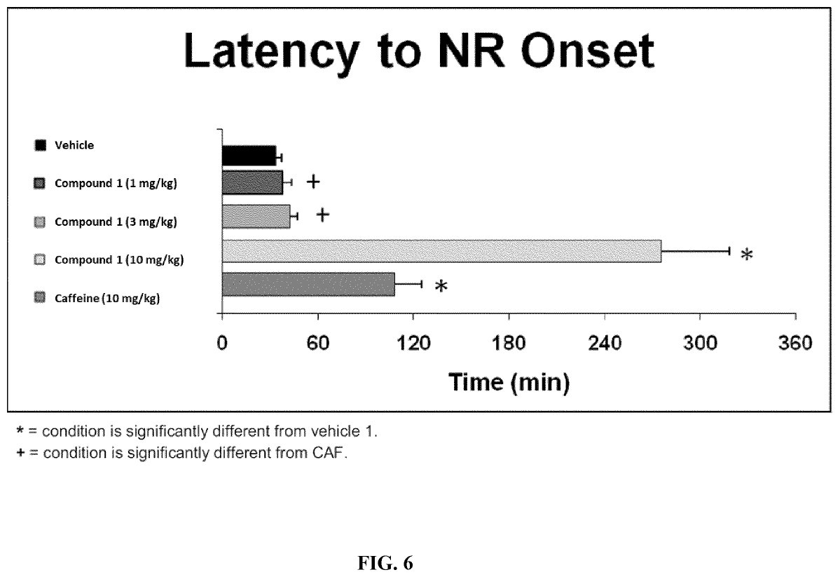

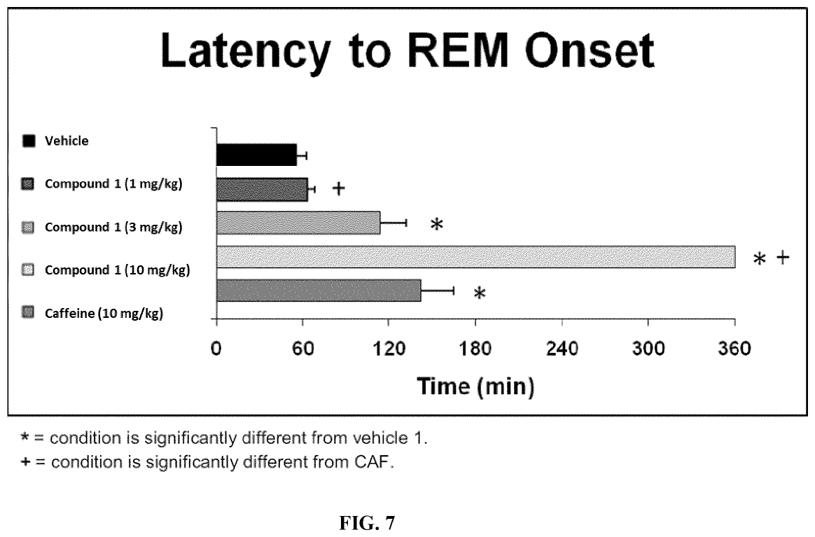

Methods. Eight (n=8) adult male Sprague Dawley rats were used in this study. Each rat received Compound 1 at 3 doses (1, 3 and 10 mg/kg), vehicle (purified H₂O-negative control) and caffeine (10 mg/kg—positive control). Dosings were administered orally during the middle of the rats' normal inactive period on occurrences separated by a minimum of 3 days. Sleep parameters (non-rapid eye movement sleep, NR; rapid eye movement sleep, REM) were recorded and the first 6 hours post-dosing records were analyzed. Results were compared to vehicle, as shown in FIG. 6 and FIG. 7.

Results. Compound 1 has wake-promoting effects at 10 mg/kg p.o. in rats with a complete suppression of REM sleep. 3 mg/kg p.o. also significantly increased wake and reduced REM sleep, but to a lesser extent.

### Example 9. Assessment of Compound 1 in the Rat 5-Choice Serial Reaction Time Task (5-CSRTT)

The 5-choice serial reaction time task (5-CSRTT) is widely used to measure attentional performance and response control (motor impulsivity) in rodents. A strength of the test is its adaptability to task modification. Variations to stimulus duration and frequency of stimulus presentation, amongst others, have become commonly used to challenge performance in the context of attention and response control. The primary objective of these studies was to examine the effect of Compound 1 on attentional performance and on aspects of response control, defined as premature responses (PREM) and perseverative responses (PSV), measures of impulsive action, and compulsive action respectively. Two experimental manipulations were undertaken to differentially challenge performance. Specifically, (1) test Compound 1 (1, 3, 10 mg/kg) under standard test conditions of 0.75 s SD, 5 s ITI, 100 trials, and (2) test Compound 1 (1, 3 mg/kg) against a long ITI challenge. The long ITI challenge is designed to elevate PREM and PSV responses and so provide a means to examine the effect of both test articles on measures of impulsive and compulsive action.

### Materials

### Vehicle Control

Dosage form: 0.3% Tween80 in 0.9% Saline
Pretreatment time: 180 minutes

### Compound 1

Dosage form: Compound 1 was suspended in 0.3% Tween80 in 0.9% Saline and sonicated. Drug was administered at a volume of 5 mL/kg, oral (PO) route.
Pretreatment time: 180 minutes
Doses tested: Phase 1: 1, 3, 10 mg/kg; Phase 2: 1, 3 mg/kg

### Subjects: 24 Male Long Evans Rats

Housing and Management of Test System: Following a one-week period of familiarization to the test facility, animals were placed on a restricted diet regimen, in which they were fed approximately 20 g of standard laboratory chow once per day (corresponding to ˜80% of normal food consumption) following the completion of study procedures (between 16:00 h and 18:00 h). Overall food intakes were therefore based on food earned during 5-choice training/testing and chow at the end of each study day. On days where no training/testing occurred, the daily allotted lab chow was increased to approximately 24 g per animal. Water was available ad-libitum except during 5-choice training/testing sessions. Animals were maintained on a 12 h/12 h light/dark cycle with all testing conducted during the animals' light cycle.

5-choice serial reaction time task: 5-choice operant chambers were housed in sound-insulated and ventilated enclosures. Chambers consisted of an aluminum enclosure (25×30 cm), containing a reward magazine attached to a food pellet dispenser and house light on one wall, and on the opposite wall an array of 5 square niches (2.5×2.5×2.5 cm) arranged on a curved panel and raised 2.5 cm from the grid floor. An LED was positioned at the rear of each niche. Each niche, and the reward magazine, also contained a photocell to detect head entry. Test chambers were controlled by Med PC software.

The 5-CSRTT schedule began with the illumination of the house light and delivery of a food pellet. A nose-poke into the magazine tray initiated the first trial which consisted of an inter-trial interval (ITI, 5 s) followed by the random illumination of one of the 5 lights for a fixed interval (stimulus duration, SD). If a nose-poke was registered in the illuminated niche before the end of either the SD, or a fixed interval after this period (limited hold, LH) a further pellet was dispensed and a Correct Trial registered. An incorrect nose poke (Incorrect Trial) or failure to respond within the allotted time (Missed Trial) resulted in a Time Out (TO) period in which the house light was extinguished for 5 s. Responding into one of the five niches during the ITI (PREM response), resulted in a further TO. PSV responses were not punished by a TO.

Each session ran for either 100 trials or 60 min, depending on which session endpoint was achieved first. Animals were trained via a series of steps to final test conditions of 0.75 s SD, 5 s ITI, 5 s limited hold. Target performance was stable performance around a threshold of 80% correct ([correct/(correct+incorrect)]*100) and <20% omissions for at least a two week period. At this point drug testing began according to a repeated measures design with animals receiving treatment over repeated test sessions. Test sessions were run twice weekly under the standard (5 s ITI) and long ITI schedule to allow drug washout and to re-baseline subjects between cycles.

Phase 1: The effect of Compound 1 (1, 3, 10 mg/kg) or vehicle control was investigated on test performance under standard test conditions of: 0.75 s SD, 5 s ITI, 5 s LH, 100 trials total. Treatments were administered in a randomized sequence.

Phase 2: The effect of Compound 1 (1 and 3 mg/kg) or vehicle control was investigated in a 10 s ITI 5-choice schedule (10 s ITI, 0.3 s SD, 5 s LH, 100 trials). Treatments were administered in a randomized sequence.

Statistical Analyses: Primary measures of performance from the 5-CSRTT, i.e. accuracy measured either as % correct ([#correct/#correct+#incorrect]*100) or % hit ([#correct/#correct+#incorrect+#omissions]*100), speed of responding (correct latency), incomplete trials (omissions) and total #trials are expressed as means and SEM.

### Results

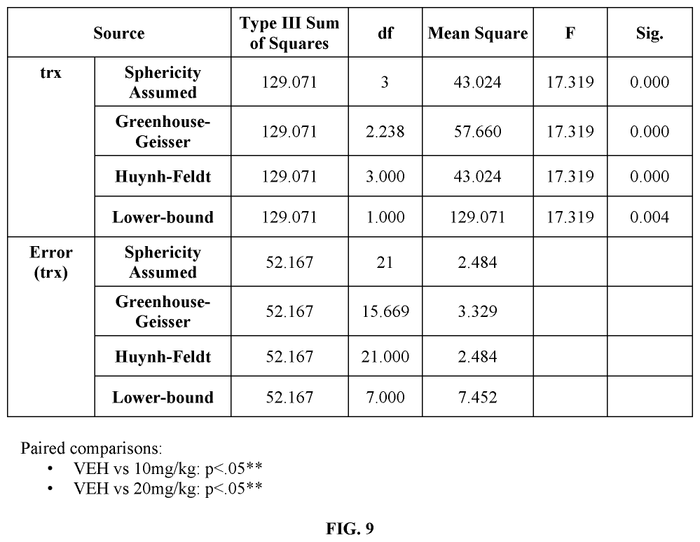

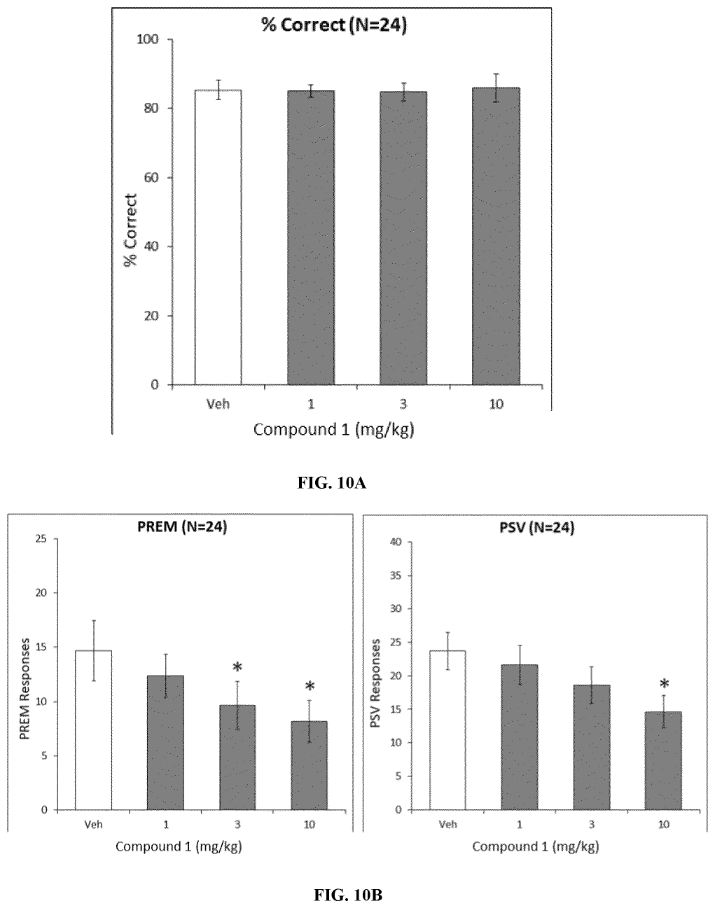

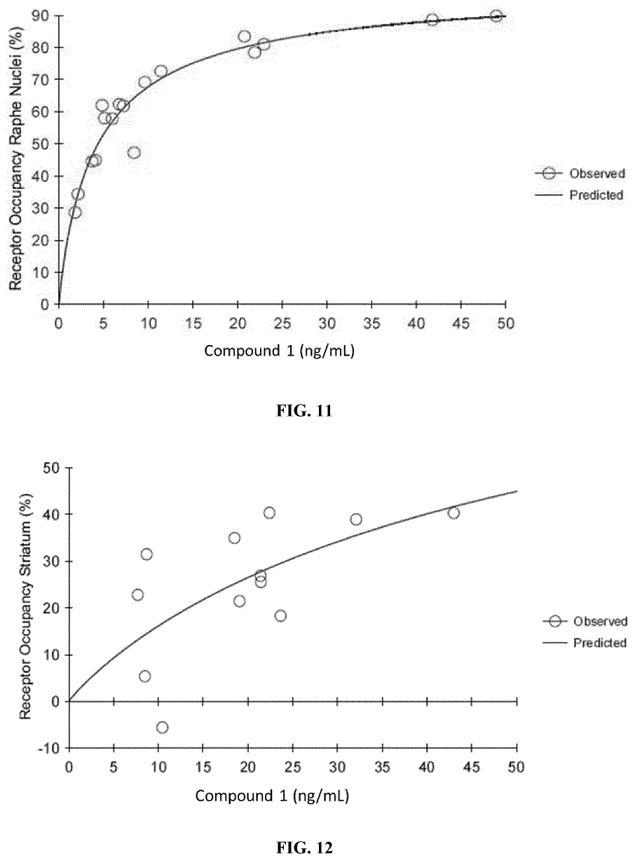

Phase 1: Effect of Compound 1 on 5-choice task performance: Standard Conditions: Compound 1 was tested at doses 1, 3, and 10 mg/kg in all rats at each dose according to a repeated measures design. A vehicle pretreated group served as control. Main effects of treatment were found for total trials (F3,69=4.4; P<0.01), PREM responses (F3,69=5.1; P<0.01) and PSV responses (F3,69=4.6; P<0.01). These main effects reflected a dose-related decrease in PREM and PSV responses following Compound 1 pretreatment relative to vehicle control. Additionally, trial number was decreased at the 10 mg/kg dose. There were no main effects of treatment on any other task measure including accuracy. 5-CSRTT results obtained under standard test conditions (0.75 sec. stimulus duration, 5 sec. ITI, 100 trials) are shown in FIG. 10A and FIG. 10B.

Phase 2: Effect of Compound 1 on 5-choice task performance: 10 s ITI: Compound 1 was tested at doses 1, and 3 mg/kg in all rats at each dose according to a repeated measures design under the 10 s ITI schedule. A vehicle pretreated group served as control. Increasing the ITI from 5 s to 10 s, and duration of the visual stimulus from 0.75 s to 0.3 s, reduced accuracy (% correct; 5 s ITI: 83.9±1.5%; 10 s ITI: 68.5±2.4%; P<0.01) and increased both PREM (5 s ITI: 6.0±1.2; 10 s ITI: 46.7±10.2; P<0.01) and PSV (5 s ITI: 20.9±3.4; 10 s ITI: 33.8±7.4; P<0.05) responses. The effect of Compound 1 on task performance under the 10 s ITI schedule was evaluated against vehicle pretreatment under the same condition. There were no main effects of treatment on any measure (F2,40≤2.5; NS), i.e., no effects of treatment on accuracy (% correct, % hit) or on PREM/PSV responses when all N=21 rats were included in the analysis. Subsequently the rats were sub-grouped into low impulsive (LI) and high impulsive (HI) based on the level of PREM responses made following vehicle pretreatment under the 10 s ITI schedule, (i.e., PREM: LI: 16.7±2.4; HI: 93.9±21.6; P<0.01). This subgrouping resulted in main effects of subgroup on PREM (F1,12=8.0; P=0.02), and PSV responses (F1,12=8.3; P=0.01), reflecting the HI rats to have significantly higher PREM and PSV responses relative to their LI counterparts. In terms of the effect of Compound 1 on task performance in these subgroups, main effects of treatment and subgroup x treatment interactions were noted for both PREM (treatment: F2,24=6.7; P<0.01; subgroup x treatment: F2,24=10.2; P<0.01) and PSV responses (treatment: F2,24=4.2; P=0.02; subgroup x treatment: F2,24=7.7; P<0.01).

### Example 10. A Randomised, Double-Blind, Placebo-Controlled, Cross-Over Multi-Centre Trial of Compound 1 in Adults with Atypical Depression

A randomized, double-blind, placebo-controlled crossover trial in which the efficacy and safety of Compound 1 is examined with a DSM-5 diagnosis of atypical depression. The trial consists of a Screening Period, a Treatment Period, and a Follow-up Period, as described in detail below. Participants may record symptoms associated with atypical depression using the eDiary and may wear the actigraphy watch for each day during the Treatment Periods as well as the Washout Period.

### Objectives and Endpoints

| | |
|---|---|
| Objectives | Endpoints |

Primary

| | |
|---|---|
| To examine the effect of | The efficacy of administration of |
| Compound 1 vs. placebo on | Compound 1 vs. placebo in participants |
| DSM-5 atypical depression | with atypical depression as measured by |
| symptoms | a pre-identified and/or modified subset |
| | of questions in a depressive symptoms |
| | questionnaire (e.g., Inventory of |
| | Depressive Symptoms - Self-Reported |
| | (IDS-SR)) |

Secondary

| | |
|---|---|
| Efficacy/Pharmacodynamics | Efficacy/Pharmacodynamics |
| To examine the temporal | The weekly efficacy of Compound 1 vs. |
| effect of Compound 1 vs. | placebo in participants with atypical |
| placebo on DSM-5 atypical | depression as measured by a pre- |
| depression symptoms | identified and/or modified subset of |
| | questions in the depressive symptoms |
| | questionnaire (e.g., IDS-SR) |
| To examine response rate | Response is defined as a reduction (e.g., |
| | 50%, 75%, or 100%) in atypical |
| | depression symptoms measured with a |
| | pre-specified and/or modified subset of |
| | a depressive symptoms questionnaire |
| | (e.g., IDS-SR) |
| Number of patients | Remission may be defined as absence |
| achieving remission | of DSM-5 Major Depressive Disorder |
| | (MDD) atypical subtype specifier, |
| | identified using an interview (e.g., Mini |
| | International Neuropsychiatric |
| | Interview (MINI-S)) |
| To examine the effect of | Change in CGI-S from baseline to |
| Compound 1 vs. placebo on | Week (placebo-corrected). |
| Clinical Global Impression - | |
| Severity (CGI-S) | |
| To examine the effect of | The effect of administration of |
| Compound 1 vs. placebo on | Compound 1 vs. placebo on sleep |
| sleep parameters | parameters as measured by a sleep scale |
| | (e.g., Epworth Sleepiness Scale (ESS) |
| | and/or objective sleep activity (e.g., |
| | total sleep time)). |
| To examine the effect of | CGI-I score after administration of |
| Compound 1 vs. placebo on | Compound 1 vs. placebo |
| Clinical Global Impression - | |
| Improvement (CGI-I) | |
| To examine the effect of | PGIC score after administration of |
| Compound 1 vs. placebo on | Compound 1 vs. placebo |
| Patient Global Impression | |
| of Change (PGI-C) | |
| To examine the effect of | A fatigue score (e.g., based on the Brief |
| Compound 1 on fatigue using | Fatigue Inventory (BFI), the Fatigue |
| a fatigue scale (e.g., the | Severity Scale (FSS), the Fatigue |
| Brief Fatigue Inventory (BFI), | Symptom Inventory (FSI), and/or the |
| the Fatigue Severity Scale | Fatigue - Short Form 10a (FACIT- |
| (FSS), the Fatigue Symptom | Fatigue-10)) after administration of |
| Inventory (FSI), and/or the | Compound 1 vs. placebo |
| Fatigue - Short Form 10a | |
| (FACIT-Fatigue-10)) | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed:

1. A method of treating a subject manifesting at least one or more behaviors selected from the group consisting of:
(a) (i) mood reactivity,
(ii) increased sleep,
(iii) hypersomnia,
(iv) leaden paralysis,
(v) long-standing pattern of interpersonal rejection sensitivity resulting in significant social or occupational impairment,
(b) (i) binge eating not associated with the regular use of inappropriate compensatory behaviors,
(ii) binge eating not occurring exclusively during the course of anorexia nervosa or bulimia nervosa,
(c) (i) recurrent inappropriate compensatory behaviors, such as self-induced vomiting; misuse of laxatives, diuretics, or other medications, in order to prevent weight gain,
(ii) binge eating and inappropriate compensatory behaviors,
(iii) self-evaluation is unjustifiability influenced by body shape and weight,
(iv) disturbance not occurring exclusively during episodes of anorexia nervosa,
(v) purging type of recurrent compensatory behavior, and
(vi) non-purging type of recurrent compensatory behavior,
(d) (i) recurrent episodes of binge eating,
(ii) eating in a discrete period of time an amount of food that is definitely larger than most people would eat in a similar period of time under similar circumstances,
(iii) A sense of lack of control over-eating during the episode,
(iv) A feeling that one cannot stop eating or control what or how much one is eating,
(v) eating much more rapidly than normal,
(vi) eating until feeling uncomfortably full,
(vii) eating large amounts of food when not feeling physically hungry,
(viii) eating alone because of feeling embarrassed by how much one is eating,
(ix) feeling disgusted with oneself, depressed or very guilty after overeating,
(x) marked distress regarding binge eating,
(e) (i) increased appetite,
(ii) hyperphagia,
(iii) significant lack of impulse control followed by guilt feelings,
(f) (i) restriction of energy intake relative to requirements leading to a significantly low body weight,
(ii) intense fear of gaining weight or becoming fat, even though underweight, and
(iii) disturbance in the way in which one's body weight or shape is experienced;
said method comprising administering a composition comprising Compound 1: 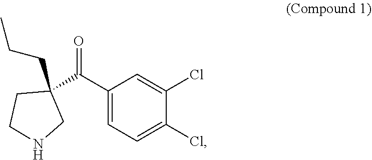 [SMILES: CCC[C@]1(C(=O)c2ccc(Cl)c(Cl)c2)CCNC1]
or a pharmaceutically acceptable salt thereof, sufficient to improve at least one of the behaviors.

2. The method of claim 1, wherein the composition is a pharmaceutical composition.

3. The method of claim 1, wherein the composition is a sustained release form.

4. The method of claim 1, wherein the composition is a unit dose.

5. The method of any one of claims 1-4, wherein the composition comprises from about 1 mg/kg to about 70 mg/kg of Compound 1, or a pharmaceutically acceptable salt thereof.

6. The method of any one of claims 1-5, wherein the composition is administered orally.

7. The method of any one of claims 1-6, wherein the composition is administered once daily.

8. The method of any one of claims 1-7, wherein said subject manifests mood reactivity; and
at least two behaviors selected from the group consisting of increased appetite, hyperphagia, increased sleep, hypersomnia, leaden paralysis, and long-standing pattern of interpersonal rejection sensitivity resulting in significant social or occupational impairment.

9. The method of claim 8, wherein said subject does not meet the criteria for melancholic depression or catatonia.

10. The method of any one of claims 1-9, wherein said subject manifests:
recurrent episodes of binge eating, eating in a discrete period of time an amount of food that is definitely larger than most people would eat in a similar period of time under similar circumstances, a sense of lack of control over eating during the episode, eating much more rapidly than normal, a feeling that one cannot stop eating or control what or how much one is eating, marked distress regarding binge eating, binge eating not associated with the regular use of inappropriate compensatory behaviors, and binge eating not occurring exclusively during the course of anorexia nervosa or bulimia nervosa; and
three or more behaviors selected from the group consisting of eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating.

11. The method of claim 1, wherein said subject manifests:
recurrent episodes of binge eating, eating in a discrete period of time an amount of food that is definitely larger than most people would eat in a similar period of time under similar circumstances, a feeling that one cannot stop eating or control what or how much one is eating, recurrent inappropriate compensatory behaviors in order to prevent weight gain, binge eating and inappropriate compensatory behaviors, self-evaluation is unjustifiability influenced by body shape and weight, and disturbance not occurring exclusively during episodes of anorexia nervosa.

12. The method of claim 11, wherein the discrete period of time is a 2-hour period, and the binge eating and inappropriate compensatory behaviors occur at least twice a week for 3 months.

13. The method of claim 8, wherein said subject further manifests one or more behaviors selected from the group consisting of:
recurrent episodes of binge eating, eating in a discrete period of time an amount of food that is definitely larger than most people would eat in a similar period of time under similar circumstances, a sense of lack of control over eating during the episode, a feeling that one cannot stop eating or control what or how much one is eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with the regular use of inappropriate compensatory behaviors, binge eating not occurring exclusively during the course of anorexia nervosa or bulimia nervosa, recurrent inappropriate compensatory behaviors in order to prevent weight gain, binge eating and inappropriate compensatory behaviors, self-evaluation is unjustifiability influenced by body shape and weight, disturbance not occurring exclusively during episodes of anorexia nervosa, purging type of recurrent compensatory behavior, and non-purging type of recurrent compensatory behavior.

14. The method of any one of claims 1-13, wherein said subject is a subject who has been diagnosed with atypical depression.

15. The method of any one of claims 1-14, wherein said subject is a subject who has been diagnosed with one or more behaviors selected from the group consisting of mood reactivity, increased appetite, hyperphagia, increased sleep, hypersomnia, leaden paralysis, and long-standing pattern of interpersonal rejection sensitivity resulting in significant social or occupational impairment.

16. The method of any one of claims 1-15, wherein said subject is a subject who has been diagnosed with binge eating disorder.

17. The method of any one of claims 1-16, wherein said subject is a subject who has been diagnosed with one or more behaviors selected from the group consisting of recurrent episodes of binge eating, eating in a discrete period of time an amount of food that is definitely larger than most people would eat in a similar period of time under similar circumstances, a sense of lack of control over eating during the episode, eating much more rapidly than normal, a feeling that one cannot stop eating or control what or how much one is eating, marked distress regarding binge eating, binge eating not associated with the regular use of inappropriate compensatory behaviors, binge eating not occurring exclusively during the course of anorexia nervosa or bulimia nervosa, eating much more rapidly than normal, eating until feeling uncomfortably full, eating large amounts of food when not feeling physically hungry, eating alone because of feeling embarrassed by how much one is eating, and feeling disgusted with oneself, depressed or very guilty after overeating.

18. The method of any one of claims 1-17, wherein said subject is a subject who has been diagnosed with bulimia nervosa.

19. The method of any one of claims 1-18, wherein said subject is a subject who has been diagnosed with one or more behaviors selected from the group consisting of rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, and non-purging type of recurrent compensatory behavior.

20. The method of any one of claims 1-19, wherein said subject is a subject who has been diagnosed with anorexia nervosa.

21. The method of any one of claims 1-20, wherein said subject is a subject who has been diagnosed with one or more behaviors selected from the group consisting of restriction of energy intake relative to requirements leading to a significantly low body weight in the context of age, sex, developmental trajectory, and physical health, intense fear of gaining weight or becoming fat, even though underweight, and disturbance in the way in which one's body weight or shape is experienced, undue influence of body weight or shape on self-evaluation, or denial of the seriousness of the current low body weight.

22. The method of claim 1, wherein said subject is a subject who has been diagnosed with depression.

23. The method of any one of claims 1-22, wherein said subject is a subject who has been treated for depression.

24. The method of claim 1, wherein the method comprises treating a subject who has been diagnosed with depression and also manifests one or more atypical depression symptoms.

25. The method of claim 24, wherein said atypical depression symptom is selected from the group consisting of mood reactivity, increased appetite, hyperphagia, increased sleep, hypersomnia, leaden paralysis, and long-standing pattern of interpersonal rejection sensitivity resulting in significant social or occupational impairment.

26. The method of any one of claims 1-25, wherein the manifestation of the behavior occurs at least once daily.

27. The method of any one of claims 1-25, wherein the manifestation of the behavior persists for at least a week.

28. The method of any one of claims 1-25, wherein the manifestation of the behavior persists for at least two weeks.

29. The method of any one of claims 1-25, wherein the manifestation of the behavior persists for at least a month.

30. The method of any one of claims 1-25, wherein the manifestation of the behavior persists for at least three months.

31. The method of any one of claims 1-25, wherein the manifestation of behavior occurs periodically.

32. A method of treating a subject manifesting an impulsive behavior, comprising administering a pharmaceutical composition comprising Compound 1: 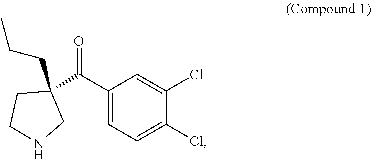 [SMILES: CCC[C@]1(C(=O)c2ccc(Cl)c(Cl)c2)CCNC1]
or a pharmaceutically acceptable salt thereof.

33. The method of claim 32, wherein the impulsive behavior is characterized by a significant lack of impulse control followed by guilt feelings.

34. A method of treating a subject who has been diagnosed with atypical depression, comprising administering a pharmaceutical composition comprising Compound 1: 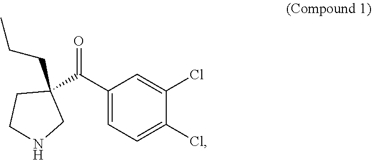 [SMILES: CCC[C@]1(C(=O)c2ccc(Cl)c(Cl)c2)CCNC1]
or a pharmaceutically acceptable salt thereof,
wherein the subject manifests one or more behavior selected from the group consisting of:
hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight.

35. A method of treating a subject manifesting at least one or more of a behavior selected from the group consisting of:
hypersomnia, hyperphagia, mood reactivity, leaden paralysis, low mood, interpersonal rejection sensitivity, rapid eating, recurrent episodes of binge eating, lack of control over eating, eating much more rapidly than normal, eating until feeling uncomfortably full, eating large amounts of food when not feeling physically hungry, eating alone because of feeling embarrassed by how much one is eating, feeling disgusted with oneself, depressed, or very guilty after overeating, marked distress regarding binge eating, binge eating not associated with compensatory behaviors, recurrent compensatory behaviors, binge eating with compensatory behavior, self-evaluation influenced by body shape and weight, purging type of recurrent compensatory behavior, non-purging type of recurrent compensatory behavior, refusal to maintain bodyweight at or above minimally normal weight, intense fear of gaining weight or becoming obese, disturbed by one's body weight or shape, self-worth influenced by body weight or shape, and persistent lack of recognition of seriousness of low bodyweight;
said method comprising administering a unit dose comprising Compound 1: 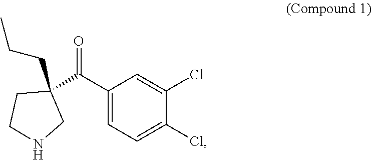 [SMILES: CCC[C@]1(C(=O)c2ccc(Cl)c(Cl)c2)CCNC1]
or a pharmaceutically acceptable salt thereof, sufficient to reduce at least of said behaviors.

36. A method of treating Prader-Willi syndrome in a subject in need thereof, comprising administering a composition comprising Compound 1: 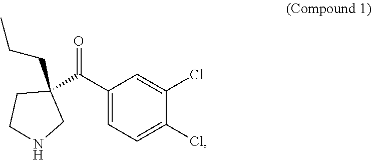 [SMILES: CCC[C@]1(C(=O)c2ccc(Cl)c(Cl)c2)CCNC1]
or a pharmaceutically acceptable salt thereof.
